(19) United States
(12) Patent Application Publication
(10) Pub. No.: US 2026/0301165 A1
(43) Pub. Date: Oct. 1, 2026

(54) SYSTEM AND METHOD FOR SEGMENTATION OF OCULAR AT-RISK AREAS

(71) Applicants: Justis P. EHLERS, Shaker Heights, OH (US); Sunil K. SRIVASTAVA, Shaker Heights, OH (US); The Cleveland Clinic Foundation, Cleveland, OH (US)

(72) Inventors: Justis P Ehlers, Shaker Heights, OH (US); Sunil K Srivastava, Shaker Heights, OH (US)

(21) Appl. No.: 19/479,243

(22) PCT Filed: Apr. 29, 2024
(86) PCT No.: PCT/US2024/026837

§ 371 Date: Oct. 27, 2025

Related U.S. Application Data
(60) Provisional application No. 63/462,821, filed on Apr. 28, 2023.

(51) Int. Cl. G06T 7/00; G06T 7/11; G16H 50/30

(52) U.S. Cl. CPC G06T 7/0012; G06T 7/11; G16H 50/30; G06T 2207/20081; G06T 2207/30041

(57) ABSTRACT

One or more computing devices, systems and/or methods are provided. In an example, a first ocular image may be evaluated to generate a first segmentation indicative of a first set of ocular zones. The first segmentation may be analyzed to identify a first set of at-risk areas associated with a risk of an eye condition. A risk mask may be generated based upon the first set of at-risk areas. A machine learning model may be trained using the risk mask to generate a trained machine learning model. A set of ocular images of an eye of a person may be received. One or more images of the set of ocular images may be evaluated using the trained machine learning model to generate an at-risk area segmentation identifying a second set of at-risk areas, of the eye, indicative of a risk of one or more eye conditions.

### RELATED APPLICATION

This application is a national stage application, filed under 35 U.S.C. § 371, of International Patent Application No. PCT/US24/026837, filed on Apr. 29, 2024, which claims priority to U.S. Provisional Patent Application No. 63/462,821, filed on Apr. 28, 2023, entitled “Automated Identification and Segmentation of Ellipsoid Zone At-Risk.” U.S. Provisional Patent Application No. 63/462,821 and International Patent Application No. PCT/US24/026837 are incorporated herein by reference in their entirety.

### TECHNICAL FIELD

The present disclosure relates to evaluating ocular images.

### BACKGROUND

Eye diseases include various conditions affecting vision, including age-related macular degeneration (AMD), diabetic retinopathy, and other conditions. Early detection of eye diseases through testing is important for managing these conditions effectively. Detecting AMD in its early stages allows for interventions that may slow its progression and preserve vision. Similarly, early detection of diabetic retinopathy enables timely treatment to prevent and/or minimize vision loss.

### SUMMARY

In accordance with the present disclosure, one or more devices, computing devices, systems and/or methods are provided. In an example, a method is provided. A first ocular image may be evaluated to generate a first segmentation indicative of a first set of ocular zones. The first segmentation may be analyzed to identify a first set of at-risk areas associated with a risk of an eye condition. A risk mask may be generated based upon the first set of at-risk areas. A machine learning model may be trained using the risk mask to generate a trained machine learning model. A set of ocular images of an eye of a person may be received. One or more images of the set of ocular images may be evaluated using the trained machine learning model to generate an at-risk area segmentation identifying a second set of at-risk areas, of the eye, indicative of a risk of one or more eye conditions.

### DESCRIPTION OF THE DRAWINGS

While the techniques presented herein may be embodied in alternative forms, the particular embodiments illustrated in the drawings are only a few examples that are supplemental of the description provided herein. These embodiments are not to be interpreted in a limiting manner, such as limiting the claims appended hereto.

FIG. 1 is an illustration of a scenario involving various examples of networks that may connect servers and clients.

FIG. 2 is an illustration of a scenario involving an example configuration of a server that may utilize and/or implement at least a portion of the techniques presented herein.

FIG. 3 is an illustration of a scenario involving an example configuration of a client that may utilize and/or implement at least a portion of the techniques presented herein.

FIG. 4A is a component block diagram illustrating training a machine learning model, in accordance with some embodiments.

FIG. 4B is a component block diagram illustrating generation of a risk mask for use in training a machine learning model, in accordance with some embodiments.

FIG. 4C illustrates an example representation of one or more determinations by a first ocular zone segmentation module and/or a risk mask generation module overlaid onto an optical coherence tomography (OCT) Brightness scan (B-scan), in accordance with some embodiments.

FIG. 4D illustrates an example representation of a risk mask overlaid onto an OCT B-scan, in accordance with some embodiments.

FIG. 4E is a component block diagram illustrating training a machine learning model, in accordance with some embodiments.

FIG. 5 is a component block diagram illustrating a system for evaluating ocular images, in accordance with some embodiments.

FIG. 6A is a component block diagram illustrating identification of one or more pathological features, in accordance with some embodiments.

FIG. 6B is a component block diagram illustrating identification of one or more pathological features, in accordance with some embodiments.

FIG. 6C is a component block diagram illustrating identification of one or more pathological features, in accordance with some embodiments.

FIG. 7 illustrates example representations associated with an at-risk area segmentation, in accordance with some embodiments.

FIG. 8 illustrates an en face at-risk area representation, in accordance with some embodiments.

FIG. 9 illustrates an example representation of at least a portion of an ocular report, in accordance with some embodiments.

FIG. 10 is a flow chart illustrating an example method, in accordance with some embodiments.

FIG. 11 is an illustration of a scenario featuring an example non-transitory machine readable medium in accordance with one or more of the provisions set forth herein.

### DETAILED DESCRIPTION

Subject matter will now be described more fully hereinafter with reference to the accompanying drawings, which form a part hereof, and which show, by way of illustration, specific example embodiments. This description is not intended as an extensive or detailed discussion of known concepts. Details that are known generally to those of ordinary skill in the relevant art may have been omitted, or may be handled in summary fashion.

The following subject matter may be embodied in a variety of different forms, such as methods, devices, components, and/or systems.

Accordingly, this subject matter is not intended to be construed as limited to any example embodiments set forth herein. Rather, example embodiments are provided merely to be illustrative. Such embodiments may, for example, take the form of hardware, software, firmware, medicine, clothing design, or any combination thereof.

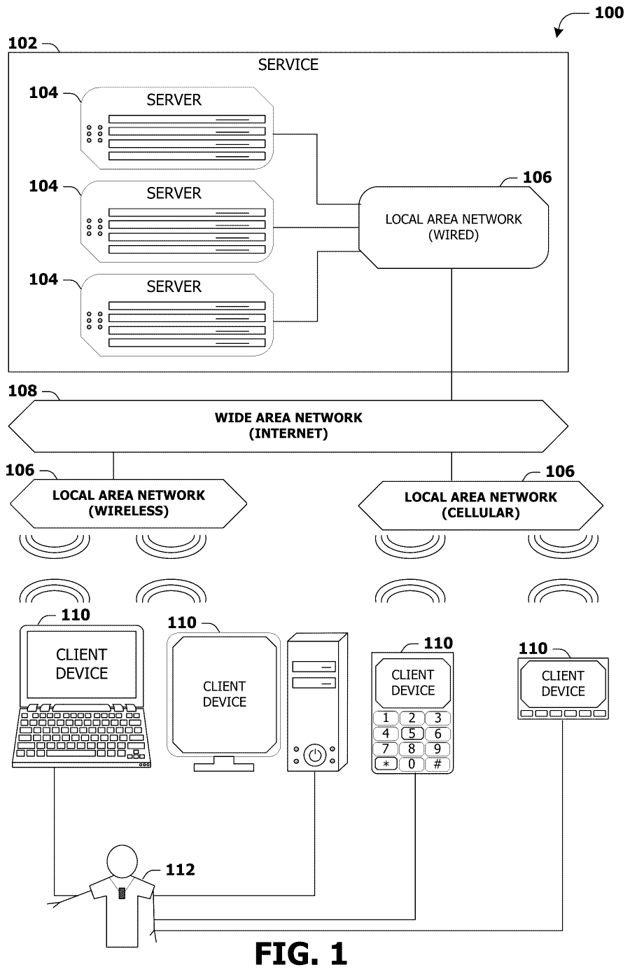

FIG. 1 is an interaction diagram of a scenario 100 illustrating a service 102 provided by a set of servers 104 to a set of client devices 110 via various types of networks. The servers 104 and/or client devices 110 may be capable of transmitting, receiving, processing, and/or storing many types of signals, such as in memory as physical memory states.

In the scenario 100 of FIG. 1, the service 102 may be accessed via a wide area network 108 (WAN) by a user 112 of one or more client devices 110, such as a portable media player (e.g., an electronic text reader, an audio device, or a portable gaming, exercise, or navigation device); a portable communication device (e.g., a camera, a phone, a wearable or a text chatting device); a workstation; and/or a laptop form factor computer. The respective client devices 110 may communicate with the service 102 via various connections to the wide area network 108.

One or more client devices 110 may comprise a cellular communicator and may communicate with the service 102 by connecting to the wide area network 108 via a wireless local area network 106 (LAN) provided by a cellular provider.

Alternatively and/or additionally, one or more client devices 110 may communicate with the service 102 by connecting to the wide area network 108 via a wireless local area network 106 provided by a location such as the user's home or workplace. The wireless local area network 106 may, for example, be a WiFi (Institute of Electrical and Electronics Engineers (IEEE) Standard 802.11) network or a Bluetooth (IEEE Standard 802.15.1) personal area network.

It may be appreciated that the servers 104 and the client devices 110 may communicate over various types of networks. Exemplary types of networks that may be accessed by the servers 104 and/or client devices 110 include mass storage, such as network attached storage (NAS), a storage area network (SAN), or other forms of computer or machine readable media.

The servers 104 of the service 102 may be interconnected directly, or through one or more other networking devices, such as routers, switches, and/or repeaters. The servers 104 may utilize a variety of physical networking protocols, such as Ethernet and/or Fiber Channel, and/or logical networking protocols, such as variants of an Internet Protocol (IP), a Transmission Control Protocol (TCP), and/or a User Datagram Protocol (UDP).

The servers 104 of the service 102 may be internally connected via a local area network 106. The local area network 106 may be organized according to one or more network architectures, such as server/client, peer-to-peer, and/or mesh architectures, and/or a variety of roles, such as administrative servers, authentication servers, security monitor servers, data stores for objects such as files and databases, business logic servers, time synchronization servers, and/or front-end servers providing a user-facing interface for the service 102.

The local area network 106 may be a wired network where network adapters on the respective servers 104 are interconnected via cables (e.g., coaxial and/or fiber optic cabling), and may be connected in various topologies (e.g., buses, token rings, meshes, and/or trees). The local area network 106 may include, e.g., analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art.

Alternatively and/or additionally, the local area network 106 may comprise one or more sub-networks, such as may employ differing architectures, may be compliant or compatible with differing protocols and/or may interoperate within the local area network 106. Additionally, a variety of local area networks 106 may be interconnected; e.g., a router may provide a link between otherwise separate and independent local area networks 106.

In the scenario 100 of FIG. 1, the local area network 106 of the service 102 is connected to a wide area network 108 that allows the service 102 to exchange data with other services 102 and/or client devices 110. The wide area network 108 may encompass various combinations of devices with varying levels of distribution and exposure, such as a public wide-area network (e.g., the Internet) and/or a private network (e.g., a virtual private network (VPN) of a distributed enterprise).

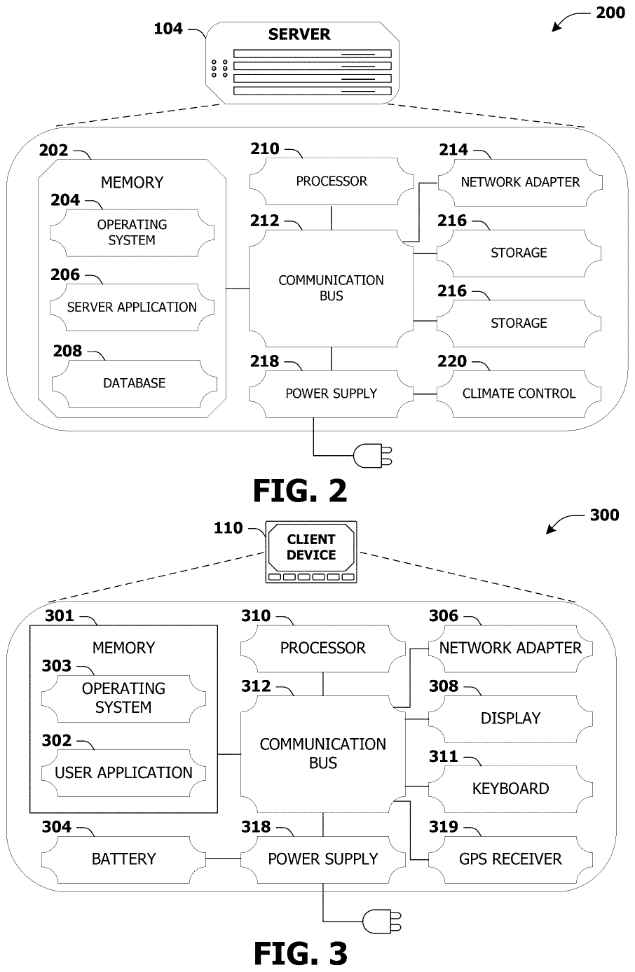

FIG. 2 presents a schematic architecture diagram 200 of a server 104 that may utilize at least a portion of the techniques provided herein. Such a server 104 may vary widely in configuration or capabilities, alone or in conjunction with other servers, in order to provide a service such as the service 102.

The server 104 may comprise a variety of peripheral components, such as a wired and/or wireless network adapter 214 connectible to a local area network and/or wide area network; one or more storage components 216, such as a hard disk drive, a solid-state storage device (SSD), a flash memory device, and/or a magnetic and/or optical disk reader.

The server 104 may comprise memory 202 storing various forms of applications, such as an operating system 204; one or more server applications 206, such as a hypertext transport protocol (HTTP) server, a file transfer protocol (FTP) server, or a simple mail transport protocol (SMTP) server; and/or various forms of data, such as a database 208 or a file system.

The server 104 may comprise one or more processors 210 that process instructions. The one or more processors 210 may optionally include a plurality of cores; one or more coprocessors, such as a mathematics coprocessor or an integrated graphical processing unit (GPU); and/or one or more layers of local cache memory.

The server 104 may comprise a mainboard featuring one or more communication buses 212 that interconnect the processor 210, the memory 202, and various peripherals, using a variety of bus technologies, such as a variant of a serial or parallel AT Attachment (ATA) bus protocol; a Uniform Serial Bus (USB) protocol; and/or Small Computer System Interface (SCI) bus protocol. In a multibus scenario, a communication bus 212 may interconnect the server 104 with at least one other server.

The server 104 may operate in various physical enclosures, such as a desktop or tower, and/or may be integrated with a display as an “all-in-one” device. The server 104 may be mounted horizontally and/or in a cabinet or rack, and/or may simply comprise an interconnected set of components.

The server 104 may provide power to and/or receive power from another server and/or other devices. The server 104 may comprise a dedicated and/or shared power supply 218 that supplies and/or regulates power for the other components. The server 104 may comprise a shared and/or dedicated climate control unit 220 that regulates climate properties, such as temperature, humidity, and/or airflow.

The server 104 may include one or more other components that are not shown in the schematic diagram 200 of FIG. 2, such as a display; a display adapter, such as a graphical processing unit (GPU); input peripherals, such as a keyboard and/or mouse; and a flash memory device that may store a basic input/output system (BIOS) routine that facilitates booting the server 104 to a state of readiness. A plurality of such servers 104 may be configured and/or adapted to utilize at least a portion of the techniques presented herein.

FIG. 3 presents a schematic architecture diagram 300 of a client device 110 whereupon at least a portion of the techniques presented herein may be implemented. Such a client device 110 may vary widely in configuration or capabilities, in order to provide a variety of functionality to a user such as the user 112.

The client device 110 may comprise memory 301 storing various forms of applications, such as an operating system 303; one or more user applications 302, such as document applications, media applications, file and/or data access applications, communication applications such as web browsers and/or email clients, utilities, and/or games; and/or drivers for various peripherals.

In some examples, as a user 112 interacts with a software application on a client device 110 (e.g., an instant messenger and/or electronic mail application), descriptive content in the form of signals or stored physical states within memory (e.g., an email address, instant messenger identifier, phone number, postal address, message content, date, and/or time) may be identified.

In such examples, descriptive content may be stored, typically along with contextual content. For example, the source of an email address (e.g., a communication received from another user via an instant messenger application) may be stored as contextual content associated with the email address. Contextual content, therefore, may identify circumstances surrounding receipt of an email address (e.g., the date or time that the email address was received), and may be associated with descriptive content.

Contextual content, may, for example, be used to subsequently search for associated descriptive content. For example, a search for email addresses received from specific individuals, received via an instant messenger application or at a given date or time, may be initiated.

The client device 110 may comprise one or more processors 310 that process instructions. The one or more processors 310 may optionally include a plurality of cores; one or more coprocessors, such as a mathematics coprocessor or an integrated graphical processing unit (GPU); and/or one or more layers of local cache memory.

The client device 110 may comprise a dedicated and/or shared power supply 318 that supplies and/or regulates power for other components, and/or a battery 304 that stores power for use while the client device 110 is not connected to a power source via the power supply 318. The client device 110 may provide power to and/or receive power from other client devices.

The client device 110 may comprise a variety of peripheral components, such as a wired and/or wireless network adapter 306 connectible to a local area network and/or wide area network; one or more output components, such as a display 308 coupled with a display adapter (optionally including a graphical processing unit (GPU)), a sound adapter coupled with a speaker, and/or a printer; input devices for receiving input from the user, such as a keyboard 311, a mouse, a microphone, a camera, and/or a touch-sensitive component of the display 308; and/or environmental sensors, such as a global positioning system (GPS) receiver 319 that detects the location, velocity, and/or acceleration of the client device 110, a compass, accelerometer, and/or gyroscope that detects a physical orientation of the client device 110.

The client device 110 may comprise a mainboard featuring one or more communication buses 312 that interconnect the processor 310, the memory 301, and various peripherals, using a variety of bus technologies, such as a variant of a serial or parallel AT Attachment (ATA) bus protocol; the Uniform Serial Bus (USB) protocol; and/or the Small Computer System Interface (SCI) bus protocol.

The client device 110 may include one or more other components that are not shown in the schematic architecture diagram 300 of FIG. 3, such as one or more storage components, such as a hard disk drive, a solid-state storage device (SSD), a flash memory device, and/or a magnetic and/or optical disk reader; and/or a flash memory device that may store a basic input/output system (BIOS) routine that facilitates booting the client device 110 to a state of readiness. In some examples, the client device 110 may include a climate control unit that regulates climate properties, such as temperature, humidity, and airflow.

The client device 110 may include one or more servers that may locally serve the client device 110 and/or other client devices of the user 112 and/or other individuals. For example, a locally installed webserver may provide web content in response to locally submitted web requests. Many such client devices 110 may be configured and/or adapted to utilize at least a portion of the techniques presented herein.

The client device 110 may serve the user in a variety of roles, such as a workstation, kiosk, media player, gaming device, and/or appliance. The client device 110 may therefore be provided in a variety of form factors, such as a desktop or tower workstation; an “all-in-one” device integrated with a display 308; a laptop, tablet, convertible tablet, or palmtop device; a wearable device mountable in a headset, eyeglass, earpiece, and/or wristwatch, and/or integrated with an article of clothing; and/or a component of a piece of furniture, such as a tabletop, and/or of another device, such as a vehicle or residence.

Geographic atrophy (GA) may be a late-stage finding in age-related macular degeneration (AMD) resulting from atrophic changes in the retinal outer layers and/or retinal pigment epithelium (RPE). Progression to GA, particularly subfoveal GA, may result in (permanent, for example) vision loss. GA may have a significant (e.g., an exponential) increase in prevalence with age. In view of the aging population, the number of individuals affected by GA may grow further in the near future. Alternatively and/or additionally, retinal toxicity (e.g., drug-related toxicity from drugs such as hydroxychloroquine which may be used as a treatment for disorders such as connective tissue disorders) may be associated with permanent and/or progressive vision loss (even after medication cessation, for example).

The irreversible nature of these diseases makes monitoring an important part of clinical care of these patients. Optical coherence tomography (OCT) may be a useful tool for screening for one or more eye conditions such as GA, retinal toxicity, etc. Spectral domain (SD)-OCT, being a three-dimensional imaging technique, may offer several benefits over two-dimensional approaches (like color fundus photography and/or fundus autofluorescence), including thorough characterization of the inner and/or outer retinal layers at high resolution. The use of an en face image may complement the standard cross sectional OCT B-scan allowing for a complete macular review at various depth levels, which may give further anatomic insight into this condition.

The ellipsoid zone (EZ) may be a hyperreflective band visualized in the outer retina on OCT that is a mitochondrial-rich of the outer part of the photoreceptor inner segments. EZ integrity has been established as a key feature of multiple degenerative retinal diseases with EZ being a surrogate for photoreceptor health. EZ disruption has been linked to visual progression, disease burden, specific diagnoses, prognosis, risk for disease severity, and treatment response to therapeutics. Patterns of EZ loss can be highly specific for diagnoses (and/or useful for screening for eye conditions such as GA, retinal toxicity, etc.). However, measuring EZ integrity manually may not be feasible and/or practical due to the high work-burden and complexity associated with that assessment. There is thus a need for an automated detection tool for detecting at-risk areas (e.g., at-risk retinal areas, such as at-risk EZ areas), which may be a biomarker for profiling disease features, disease progression, disease burden, diagnostic features, prognostic risk, and/or therapeutic response prediction.

Accurate and/or reproducible automated detection and/or quantification of at-risk areas may facilitate clinician identification of patients who may benefit from therapy and/or may enable readily screening for early detection of an eye condition (e.g., detection during an early stage of AMD prior to irreversible progression to complete GA), retinal toxicity, etc. Thus, an automated method for identifying, segmenting, and/or quantifying at-risk areas may be beneficial for monitoring patients in clinical practice and/or quantifying the effectiveness of novel treatments in clinical studies, and/or may have the capacity to swiftly and/or reliably extract measurable structural properties at-risk areas that can aid in clinical decision making.

Thus, in accordance with some embodiments, automated approaches to identifying at-risk areas (e.g., at-risk retinal areas) and/or determining risk classifications associated with the at-risk areas are provided herein. In an example, a first ocular image may be evaluated to generate a first segmentation indicative of a first set of ocular zones. The first segmentation may be analyzed to identify a first set of at-risk areas associated with a risk of an eye condition. A risk mask may be generated based upon the first set of at-risk areas. A machine learning model may be trained using the risk mask to generate a trained machine learning model. A set of ocular images of an eye of a person may be received. One or more images of the set of ocular images may be evaluated using the trained machine learning model to generate an at-risk area segmentation identifying a second set of at-risk areas, of the eye, indicative of a risk of one or more eye conditions.

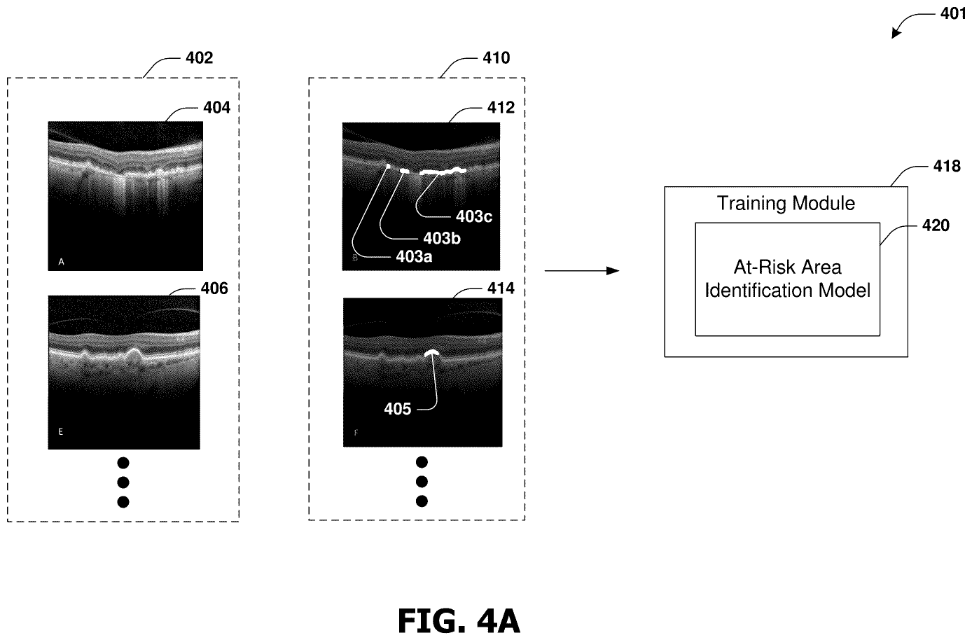

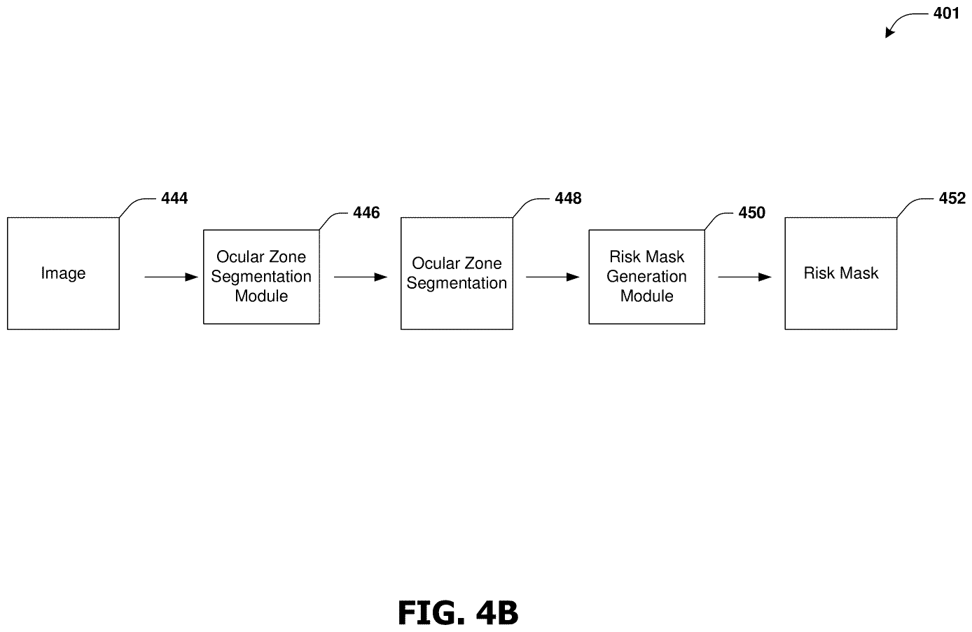

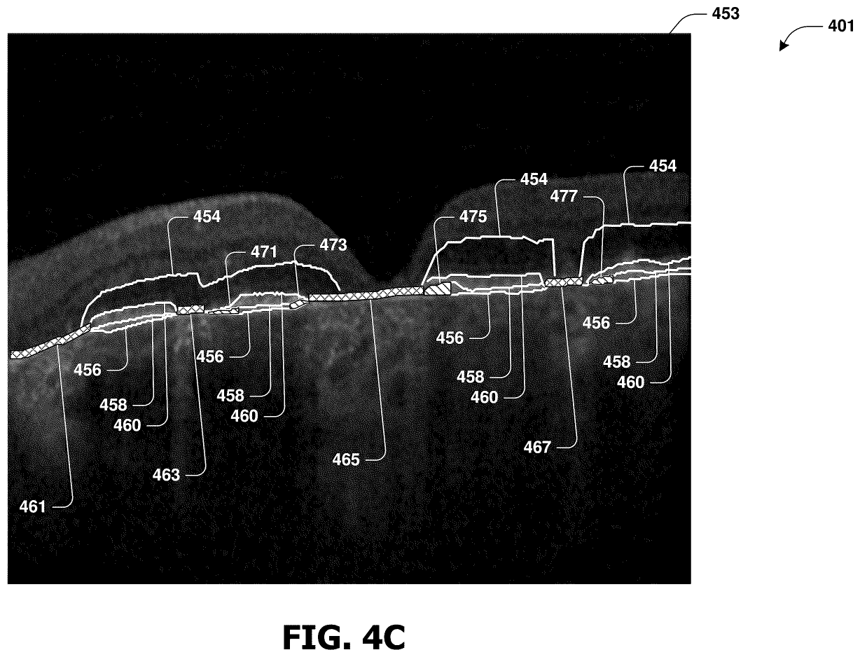

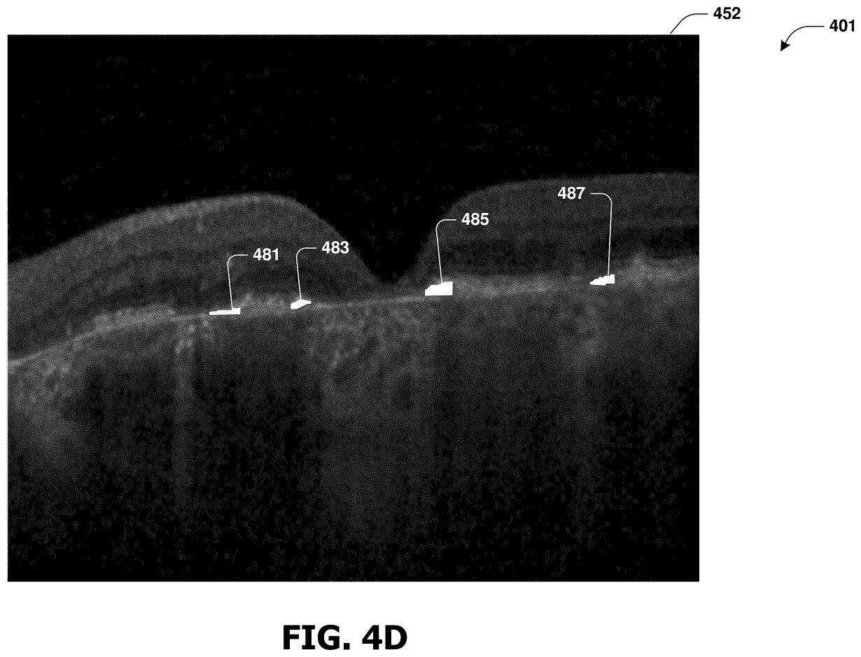

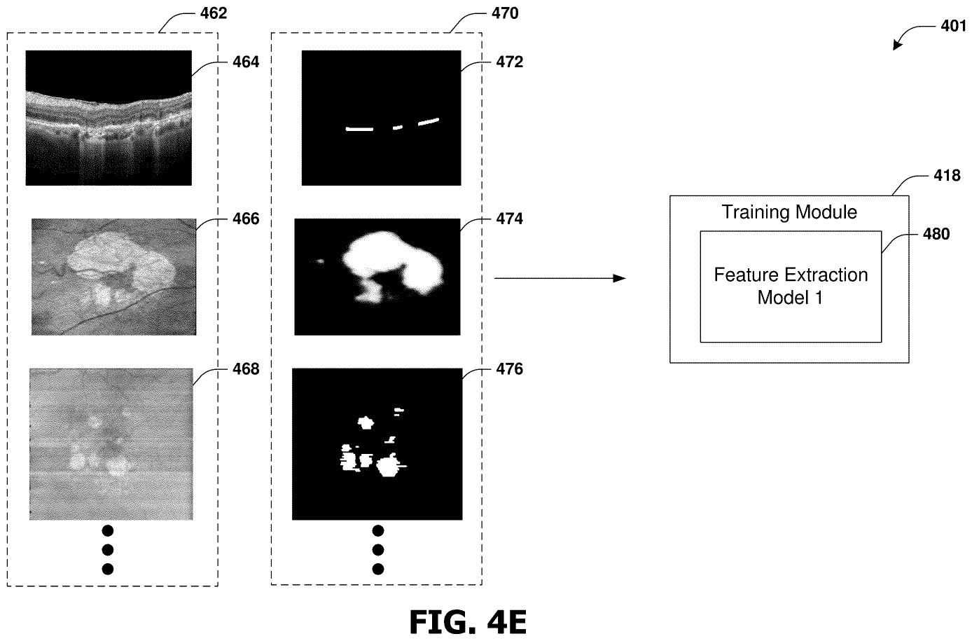

FIGS. 4A-4D illustrate a machine learning model training system 401 training machine learning models of a multi-model ocular evaluation system, in accordance with some embodiments. FIG. 4A illustrates using a training module 418 to train a machine learning model to generate a trained at-risk area identification model 420. In some examples, the training module 418 trains the machine learning model to generate the trained at-risk area identification model 420 using a first plurality of images 402 and/or a first plurality of risk masks 410 (e.g., ground truth masks) associated with the first plurality of images 402. The first plurality of images 402 may comprise ocular images of a plurality of persons (e.g., persons that are diagnosed with one or more eye conditions and/or persons that are not diagnosed with any eye condition). In some examples, the first plurality of images 402 may comprise optical coherence tomography (OCT) images (e.g., OCT Brightness scans (B-scans) and/or Amplitude scans (A-scans)), such as spectral domain OCT (SD-OCT) images (e.g., SD-OCT B-scans and/or A-scans). In some examples, an OCT B-scan may comprise a cross-sectional image of a tissue of interest. An OCT B-scan may be a combination of a plurality of OCT A-scans (e.g., the sum of OCT A-scans) which creates the cross-sectional image of a tissue of interest.

In some examples, the first plurality of images 402 may comprise en face images (e.g., en face OCT images). In some examples, a plurality of OCT images (e.g., a plurality of OCT B-scans) are reconstructed to generate an en face image. In some examples, the plurality of OCT images may be associated with a macular region (e.g., an OCT macular cube and/or rectangular prism) of an eye. For example, the plurality of OCT images may comprise B-scans corresponding to cross-sectional views, of the eye, throughout the macular region. The macular region may correspond to at least a portion of the eye. In some examples, the en face image may be generated by computing an en face projection of at least a portion of the macular region. For example, the en face image may comprise an en face projection of an entirety of the macular region (without any custom segmentation slabs, for example). Alternatively and/or additionally, the en face image may be generated based upon one or more targeted areas of interest, such as at least one of a subretinal slab, a retinal slab, a sub-RPE slab, a boundary-specific slab that encompasses one or more ocular zones (e.g., one or more anatomic layers of interest), etc. For example, the en face image may be generated to comprise an en face projection of the one or more targeted areas of interest of the macular region. The en face image may comprise a visual representation of the one or more targeted areas of interest over two lateral dimensions (rather than a single lateral dimension corresponding to an OCT B-scan, for example).

In some examples, the first plurality of images 402 may be adjusted (prior to being used for machine learning model training, for example), such as at least one of resized, cropped, compressed, etc., such that images of the first plurality of images 402 have at least one of the same size, the same number of pixels, the same dimensions, etc. In an example, each image of one, some or all of the first plurality of images 402 may be automatically adjusted to have the same size (e.g., 128×128 pixels, 512×512 pixels, and/or 256×256 pixels).

In some examples, the trained at-risk area identification model 420 is trained to perform an at-risk area identification task comprising identifying one or more at-risk areas within an image (e.g., distinguish one or more segments of the image that correspond to the one or more at-risk areas from the rest of the image). An at-risk area identified by the trained at-risk area identification model 420 may be associated with (e.g., may indicate and/or serve as a biomarker for) a risk of one or more eye conditions (e.g., current disease, future disease, and/or degenerative changes), such as at least one of geographic atrophy (GA), retinal toxicity, hydroxychloroquine toxicity, chloroquine toxicity, hydroxychloroquine retinopathy, chloroquine retinopathy, macular degeneration, age-related macular degeneration (AMD), wet AMD, dry AMD, diabetic retinopathy, diabetic macular edema (DME), an inherited retinal disease, an atrophic eye disease, an inflammatory infectious disease, retinitis pigmentosa, Stargardt disease, and/or one or more other types of eye conditions. For example, the at-risk area may correspond to an area (e.g., a retinal area of a retina, an outer retinal area of an outer retinal zone, and/or an ellipsoid zone (EZ) area of an EZ, etc.) that is associated with at least one of an abnormality, photoreceptor loss, photoreceptor risk, EZ loss, EZ disruption, etc.

In some examples, masks of the first plurality of risk masks 410 may identify segments of respective images of the first plurality of images 402 that correspond to at-risk areas. The first plurality of images 402 may comprise an image 404 (e.g., a B-scan), an image 406 (e.g., a B-scan) and/or one or more other images. The first plurality of risk masks 410 may comprise (i) a risk mask 412 identifying one or more segments (e.g., segments 403a, 403b and/or 403c shown in white in FIG. 4A), of the image 404, corresponding to one or more at-risk areas (e.g., one or more areas associated with at least one of a risk of one or more eye conditions, an abnormality, photoreceptor loss, photoreceptor risk, EZ loss, EZ disruption, etc.), and/or (ii) a risk mask 414 identifying one or more segments (e.g., segment 405 shown in white in FIG. 4A), of the image 406, corresponding to one or more at-risk areas.

FIG. 4B illustrates generation of a risk mask 452 (e.g., a risk mask of the first plurality of risk masks 410 used to train the trained at-risk area identification model 420) using a first ocular zone segmentation module 446 and/or a risk mask generation module 450. In an example, the first ocular zone segmentation module 446 may evaluate an image 444 (e.g., an image of the first plurality of images 402) to generate a first segmentation 448 (e.g., multi-layer segmentation) indicative of a first set of segments (e.g., a set of one or more segments) corresponding to a first set of ocular zones (e.g., a set of one or more ocular zones) of an eye of a person. The first set of segments may comprise line segmentations corresponding to boundaries of ocular zones and/or region of interest segmentations corresponding to regions associated with ocular zones. The first set of segments may comprise a first segment (e.g., a line segmentation and/or region of interest segmentation) corresponding to a first ocular zone of the first set of ocular zones, a second segment (e.g., a line segmentation and/or region of interest segmentation) corresponding to a second ocular zone of the first set of ocular zones, a third segment (e.g., a line segmentation and/or region of interest segmentation) corresponding to a third ocular zone of the first set of ocular zones, a fourth segment (e.g., a line segmentation and/or region of interest segmentation) corresponding to a fourth ocular zone of the first set of ocular zones, and/or one or more other segments (e.g., line segmentations and/or region of interest segmentations) corresponding to one or more other ocular zones. The first set of ocular zones may comprise at least one of at least one of an internal limiting membrane (ILM), a Bruch's membrane (BM), a retinal pigment epithelium (RPE), an outer nuclear layer (ONL), an ellipsoid zone (EZ), photoreceptor outer segments (POS), an external limiting membrane (ELM), an outer plexiform layer (OPL), an inner nuclear layer (INL), an inner plexiform layer (IPL), a ganglion cell layer (GCL), a retinal nerve fiber layer (RNFL), etc.

The first segmentation 448 may be provided to the risk mask generation module 450. The risk mask generation module 450 may (i) evaluate the first segmentation 448 to identify a first set of at-risk areas (e.g., a set of one or more at-risk areas) associated with a risk of one or more eye conditions and/or (ii) generate the risk mask 452 based upon the one or more portions. In some examples, the first set of at-risk areas may comprise a set of at-risk portions (e.g., a set of one or more at-risk portions), of the first set of ocular zones, associated with a risk of the one or more eye conditions FIG. 4C illustrates an example representation 453 indicative of one or more determinations by the first ocular zone segmentation module 446 and/or the risk mask generation module 450. The example representation 453 may be indicative of one or more segments of the first set of segments of the first segmentation 448, such as the first segment (shown with reference number 460) corresponding to ellipsoid zone (EZ) (e.g., the first ocular zone corresponds to at least a portion of EZ), the second segment (shown with reference number 458) corresponding to retinal pigment epithelium (RPE) (e.g., the second ocular zone corresponds to at least a portion of RPE), the third segment (shown with reference number 456) corresponding to Bruch's membrane (BM) (e.g., the third ocular zone corresponds to at least a portion of BM) and/or the fourth segment (shown with reference number 454) corresponding to outer nuclear layer (ONL) (e.g., the fourth ocular zone corresponds to at least a portion of ONL)).

In some examples, the risk mask generation module 450 may (i) determine the first set of at-risk areas and/or the set of at-risk portions based upon the first segmentation 448 (e.g., based upon the first set of segments 460, 458, 456 and/or 454), and/or (ii) generate the risk mask 452 based upon the first set of at-risk areas and/or the set of at-risk portions. In an example, the risk mask generation module 450 may (i) analyze the first segmentation 448 to determine a plurality of EZ-RPE thicknesses at various portions of the first set of ocular zones, (ii) compare the plurality of EZ-RPE thicknesses with a first defined threshold EZ-RPE thickness and/or a first defined range of EZ-RPE thicknesses, (iii) determine the set of at-risk portions based upon the comparisons, and/or (iv) mark the first set of at-risk areas as at-risk in the risk mask 452 based upon the set of at-risk portions (e.g., each at-risk area of the first set of at-risk areas may be generated based upon an at-risk portion, of the first set of ocular zones, of the set of at-risk portions). In some examples, the risk mask generation module 450 may identify an at-risk portion and/or may include the at-risk portion in the set of at-risk portions based upon a determination that an EZ-RPE thickness (of the plurality of EZ-RPE thicknesses) at the at-risk portion is less than the first defined threshold EZ-RPE thickness and/or within the first defined range of EZ-RPE thicknesses.

In some examples, in response to determining that an EZ-RPE thickness (of the plurality of EZ-RPE thicknesses) at a portion of the first set of ocular zones is greater than the first defined threshold EZ-RPE thickness and/or outside the first defined range of EZ-RPE thicknesses, the risk mask generation module 450 may not include the portion in the set of at-risk portions. In some examples, an EZ-RPE thickness of the plurality of EZ-RPE thicknesses corresponds to a distance between EZ (represented by the first segment 460) and RPE (represented by the second segment 458).

Accordingly, the risk mask generation module 450 may compute the plurality of EZ-RPE thicknesses by measuring distances between EZ (e.g., the first ocular zone represented by the first segment 460) and RPE (e.g., the second ocular zone represented by the second segment 458), which may be performed based upon the first segment 460 and/or the second segment 458. The first defined threshold EZ-RPE thickness may correspond to a defined threshold distance between EZ and RPE. The first defined range of EZ-RPE thicknesses may correspond to a defined range of distances between EZ and RPE. In some examples, by identifying the set of at-risk portions and/or the first set of at-risk areas using one or more of the techniques provided herein, the set of at-risk portions and/or the first set of at-risk areas may be representative of pre-GA areas of outer retinal attenuation (e.g., EZ attenuation). Embodiments are contemplated in which one or more other types of measurements (e.g., distances, thicknesses, and/or other types of measurements other than EZ-RPE thicknesses) associated with one or more other types of ocular zones (e.g., retinal zones and/or ocular zones other than EZ and/or RPE, such as at least one of ILM, BM, ONL, POS, ELM, OPL, INL, IPL, GCL, RNFL, etc.) are computed by the risk mask generation module 450 based upon the first segmentation 448 (e.g., based upon the first set of segments 460, 458, 456 and/or 454), and the measurements are used to identify the set of at-risk portions and/or the first set of at-risk areas (for use in generating the risk mask 452, for example).

In some examples, the first defined threshold EZ-RPE thickness may be between about 5 micrometers to about 20 micrometers (e.g., the first defined threshold EZ-RPE thickness may be about 10 micrometers) and/or other value. In some examples, the first defined range of EZ-RPE thicknesses may range from at least about 2 micrometers to at most about 20 micrometers, may range from at least about 2 micrometers to at most about 10 micrometers, and/or may be another range of values. In comparison, EZ-RPE thickness of a healthy and/or normal eye may be higher than 30 micrometers (e.g., between about 40 micrometers to about 50 micrometers).

The example representation 453 may be indicative of segments 471, 473, 475 and/or 477 (shown in FIG. 4C with diagonal stripe pattern-filled regions) corresponding to the first set of at-risk areas. For example, each segment of segments 471, 473, 475 and/or 477 may correspond to an at-risk area, of the first set of at-risk areas, in which an EZ-RPE thickness (e.g., a distance between EZ and RPE) is less than the first defined threshold EZ-RPE thickness and/or within the first defined range of EZ-RPE thicknesses.

In some examples, the risk mask generation module 450 may use the first segmentation 448 (e.g., the first set of segments 460, 458, 456 and/or 454) to determine a set of complete geographic atrophy (GA) portions, of the first set of ocular zones, associated with complete GA. The example representation 453 may be indicative of segments 461, 463, 465 and/or 467 (shown in FIG. 4C with diamond pattern-filled regions) corresponding to the set of complete GA portions. In some examples, complete GA may be defined by confluence of EZ (represented by the first segment 460), RPE (represented by the second segment 458) and/or BM (represented by the third segment 456). Alternatively and/or additionally, an area of complete GA may correspond to an area where (i) EZ thickness of the EZ of an eye is about 0 or less than a threshold EZ thickness, (ii) RPE thickness of the RPE of the eye is about 0 or less than a threshold RPE thickness, and/or (iii) BM thickness of the BM of the eye is about 0 or less than a threshold BM thickness. The risk mask generation module 450 may identify complete GA based upon identification of an area where EZ, RPE and/or BM are confluent (e.g., an area where some or all of EZ, RPE and/or BM overlap with each other). In some examples, the risk mask generation module 450 may exclude the set of complete GA portions from the risk mask 452 (and/or from the set of at-risk portions). For example, the risk mask 452 may be generated to exclude representations of the set of complete GA portions. Alternatively and/or additionally, the set of at-risk portions may be determined such that an at-risk portion of the set of at-risk portions does not overlap with a complete GA portion of the set of complete GA portions. Alternatively and/or additionally, the first set of at-risk areas may be determined such that an at-risk area of the first set of at-risk areas does not overlap with a complete GA portion of the set of complete GA portions.

FIG. 4D illustrates an example of the risk mask 452 generated based upon the first set of at-risk areas. For example, the risk mask 452 may be indicative of segment 481 (shown in white in FIG. 4D) corresponding to an at-risk area of the first set of at-risk areas, segment 483 (shown in white in FIG. 4D) corresponding to an at-risk area of the first set of at-risk areas, segment 485 (shown in white in FIG. 4D) corresponding to an at-risk area of the first set of at-risk areas and/or segment 487 (shown in white in FIG. 4D) corresponding to an at-risk area of the first set of at-risk areas. In some examples, the risk mask 452 may be diluted to a thickness of a defined quantity of pixels (e.g., about 10 pixels or other quantity of pixels) centered at an ocular zone (e.g., the EZ segmentation line 460) to achieve contextual mask enrichment from adjacent contiguous regions of the first set of at-risk areas.

FIG. 4E illustrates training a machine learning model to generate a first trained feature extraction model 480. In some examples, the training module 418 trains the machine learning model to generate the first trained feature extraction model 480 using a second plurality of images 462 and/or a plurality of ground truth masks 470 associated with the second plurality of images 462. The second plurality of images 462 may comprise ocular images of a plurality of persons (e.g., persons that are diagnosed with one or more eye conditions and/or persons that are not diagnosed with any eye condition). For example, the second plurality of images 462 may comprise OCT images (e.g., OCT B-scans and/or A-scans), such as SD-OCT images (e.g., SD-OCT B-scans and/or A-scans). Alternatively and/or additionally, the second plurality of images 462 may comprise en face images (e.g., en face OCT images). In some examples, a plurality of OCT images (e.g., a plurality of OCT B-scans) are reconstructed to generate an en face image of the second plurality of images 462. In some examples, the second plurality of images 462 may be adjusted (prior to being used for machine learning model training, for example), such as at least one of resized, cropped, compressed, etc., such that images of the second plurality of images 462 have at least one of the same size, the same number of pixels, the same dimensions, etc.

In some examples, the first trained feature extraction model 480 is trained to perform a feature extraction task comprising identifying one or more pathological features within an image (e.g., distinguish one or more segments of the image that correspond to the one or more pathological features from the rest of the image). In some examples, the first trained feature extraction model 480 is trained to identify one or more first pathological feature types. In some examples, the one or more first pathological feature types comprise at least one of drusen (e.g., extracellular deposits of lipids, proteins, and/or cellular debris found within one or more layers of a retina), geographic atrophy (GA), a hypertransmission defect, a hypotransmission defect, inflammatory lesion, Subretinal material (SRMat), subretinal hyperreflective material (SHRM), ellipsoid zone (EZ) loss, intraretinal fluid (IRF) (e.g., longitudinal IRF), subretinal fluid (SRF) (e.g., longitudinal SRF), cystic fluid, general fluid, sub-RPE fluid, a lesion, inflammatory debris, region with presence of new material and/or new lesion, region with absence of material and/or lesion, region of thinning, region with change in layer thickness, etc.

In some examples, masks of the plurality of ground truth masks 470 may identify respective segments of the second plurality of images 462 that correspond to pathological features of the one or more first types of pathological features. The second plurality of images 462 may comprise an image 464 (e.g., an OCT B-scan), an image 466 (e.g., an en face OCT image), an image 468 (e.g., an en face OCT image) and/or one or more other images. In an example, the one or more first types of pathological features comprise geographic atrophy (GA). The plurality of ground truth masks 470 may comprise (i) a ground truth mask 472 identifying one or more segments, of the image 464, corresponding to GA, (ii) a ground truth mask 474 identifying one or more segments, of the image 466, corresponding to GA, and/or (iii) a ground truth mask 476 identifying one or more segments, of the image 468, corresponding to GA.

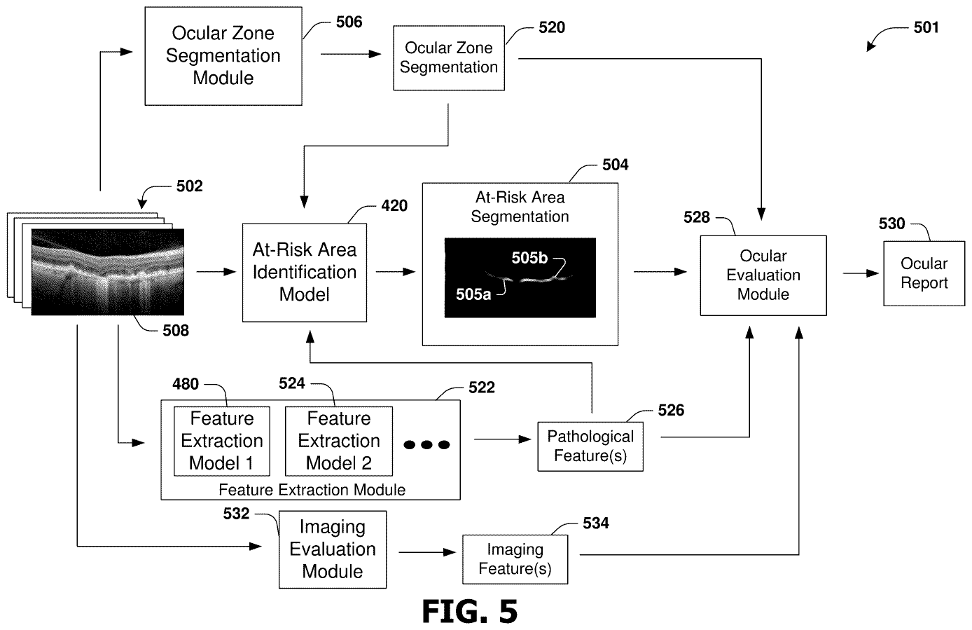

FIG. 5 illustrates a system 501 for evaluating a set of (one or more) images 502 of a first eye of a first person (e.g., a patient). The system 501 may comprise the trained at-risk area identification model 420, a second ocular zone segmentation module 506 and/or a feature extraction module 522. In some examples, the set of images 502 may comprise one or more OCT images (e.g., OCT B-scans and/or A-scans), such as SD-OCT images (e.g., SD-OCT B-scans and/or A-scans). Alternatively and/or additionally, the set of images 502 may comprise one or more en face images (e.g., en face OCT images).

In some examples, the trained at-risk area identification model 420 may evaluate one or more images (e.g., at least one of an OCT B-scan, an en face OCT image, etc.) of the set of images 502 to generate an at-risk area segmentation 504 identifying a second set of at-risk areas (e.g., a set of one or more at-risk areas), of the first eye, indicative of a risk of one or more eye conditions. For example, the trained at-risk area identification model 420 may perform the at-risk area identification task on a first image 508 (e.g., a first OCT B-scan) of the set of images 502 to generate the at-risk area segmentation 504. The at-risk area segmentation 504 may comprise a segment 505a corresponding to a first at-risk area of the first eye and/or a segment 505b corresponding to a second at-risk area of the first eye. For example, the segment 505a may identify a section, of the first image 508, corresponding to the first at-risk area and/or the segment 505b may identify a section, of the first image 508, corresponding to the second at-risk area.

Accordingly, the at-risk area segmentation 504 may indicate that the first at-risk area (represented by the segment 505a) and/or the second at-risk area (represented by the segment 505b) are associated with a risk of one or more eye conditions (e.g., a comparatively higher risk of the one or more eye conditions relative to one or more neighboring areas) of a first set of eye conditions comprising at least one of drusen, GA, a hypertransmission defect, a hypotransmission defect, inflammatory lesion, SRMat, SHRM, EZ loss, IRF, SRF, cystic fluid, general fluid, sub-RPE fluid, a lesion, inflammatory debris, region with presence of new material and/or new lesion, region with absence of material and/or lesion, region of thinning, region with change in layer thickness, etc. In some examples, the second set of at-risk areas identified by the at-risk area segmentation 504 may span one or more areas of the first eye. For example, an at-risk area of the second set of at-risk areas may span and/or overlap with (i) at least a portion a retina of the first eye, (ii) at least a portion of an outer retinal zone of the first eye, (iii) at least a portion of ellipsoid zone (EZ) of the first eye, (iv) at least a portion of retinal pigment epithelium (RPE) of the first eye, (v) one or more photoreceptors between the EZ and the RPE of the first eye, and/or (vi) one or more other areas of the first eye. Although the trained at-risk area identification model 420 may be trained using risk masks (e.g., the first plurality of risk masks 410) that are generated based upon the first set of ocular zones, the at-risk area identification model 420 may contextually evaluate regions (of the first image 508, for example) outside the first set of ocular zones to perform the at-risk area identification task and/or detect one or more at-risk areas (that the trained at-risk area identification model 420 determines are linked to risk for one or more of the first set of eye conditions, for example) based upon visual characteristics of the regions. Accordingly, in some examples, one or more of the set of at-risk regions may be at least partially outside the first set of ocular zones. In the example shown in FIG. 5, the at-risk area segmentation 504 may identify at-risk EZ areas associated with the first eye. An at-risk EZ area may comprise a portion, of the EZ of the first eye, that is associated with (i) photoreceptor loss exceeding a threshold photoreceptor loss and/or (ii) thinning of the EZ and/or the RPE to a thickness less than a threshold thickness), which may be reflective of a risk of one or more of the first set of eye conditions.

In some examples, the second ocular zone segmentation module 506 may evaluate one or more images (e.g., at least one of an OCT B-scan, an en face OCT image, the first image 508, etc.) of the set of images 502 to generate a second segmentation 520 (e.g., a multi-layer ocular segmentation profile) associated with the first eye of the first person. In some examples, the second ocular zone segmentation module 506 may be the same as or different than the first ocular zone segmentation module 446. The second segmentation 520 may be indicative of a second set of segments (e.g., a set of one or more segments) corresponding to a second set of ocular zones (e.g., a set of one or more ocular zones) of the first eye. The second set of segments may comprise line segmentations corresponding to boundaries of ocular zones and/or region of interest segmentations corresponding to regions associated with ocular zones. The second set of segments may comprise a fifth segment (e.g., a line segmentation and/or region of interest segmentation) corresponding to a fifth ocular zone of the second set of ocular zones (which may be the same as or different than the first ocular zone of the first set of ocular zones), a sixth segment (e.g., a line segmentation and/or region of interest segmentation) corresponding to a sixth ocular zone of the second set of ocular zones (which may be the same as or different than the second ocular zone of the first set of ocular zones), a seventh segment (e.g., a line segmentation and/or region of interest segmentation) corresponding to a seventh ocular zone of the second set of ocular zones (which may be the same as or different than the third ocular zone of the first set of ocular zones), an eighth segment (e.g., a line segmentation and/or region of interest segmentation) corresponding to an eighth ocular zone of the second set of ocular zones (which may be the same as or different than the fourth ocular zone of the first set of ocular zones), and/or one or more other segments (e.g., line segmentations and/or region of interest segmentations) corresponding to one or more other ocular zones. The second set of ocular zones may comprise at least one of an ILM, a BM, a RPE, an ONL, an EZ, POS, ELM, an OPL, an INL, an IPL, a GCL, a RNFL, etc.

In some examples, the feature extraction module 522 is configured to perform one or more feature extraction tasks on one or more images (e.g., at least one of an OCT B-scan, an en face OCT image, the first image 508, etc.) of the set of images 502 to determine a set of (one or more) pathological features 526 of the first eye. In some examples, each feature of one, some or all of the set of pathological features 526 may be associated with a defined impact on one or more ocular zones (e.g., one or more ocular layers of the first eye of the first person) and/or one or more retinal compartment zones (e.g., one or more retinal compartment layers of the first eye of the first person). In some examples, the feature extraction module 522 may comprise a plurality of feature extraction models for detecting different pathological feature types. For example, the one or more feature extraction tasks may comprise (i) the first feature extraction task performed using the first trained feature extraction model 480 for detecting the one or more first pathological feature types, (ii) a second feature extraction task performed using a second trained feature extraction model 524 (e.g., a model, of the feature extraction module 522, trained using one or more of the techniques provided herein with respect to the first trained feature extraction model 480) for detecting one or more second pathological feature types (e.g., at least one of drusen, GA, a hypertransmission defect, a hypotransmission defect, inflammatory lesion, SRMat, SHRM, EZ loss, IRF, SRF, cystic fluid, general fluid, sub-RPE fluid, a lesion, inflammatory debris, region with presence of new material and/or new lesion, region with absence of material and/or lesion, region of thinning, region with change in layer thickness, etc.), (iii) a third feature extraction task performed using a third trained feature extraction model (e.g., a model, of the feature extraction module 522, trained using one or more of the techniques provided herein with respect to the first trained feature extraction model 480) for detecting one or more third pathological feature types (e.g., at least one of drusen, GA, a hypertransmission defect, a hypotransmission defect, inflammatory lesion, SRMat, SHRM, EZ loss, IRF, SRF, cystic fluid, general fluid, sub-RPE fluid, a lesion, inflammatory debris, region with presence of new material and/or new lesion, region with absence of material and/or lesion, region of thinning, region with change in layer thickness, etc.), and/or (iv) one or more other feature extraction tasks performed using one or more other models of the feature extraction module 522 for detecting other pathological feature types.

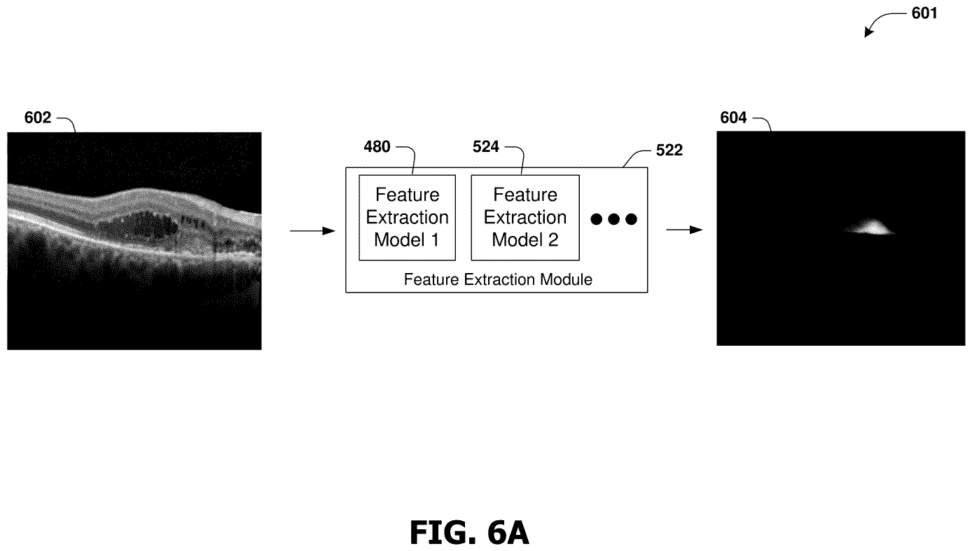

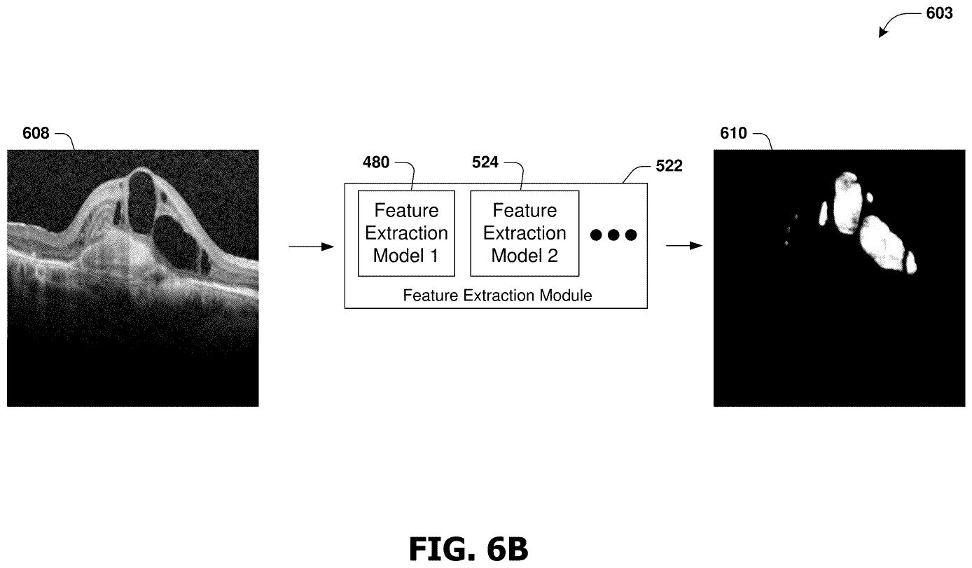

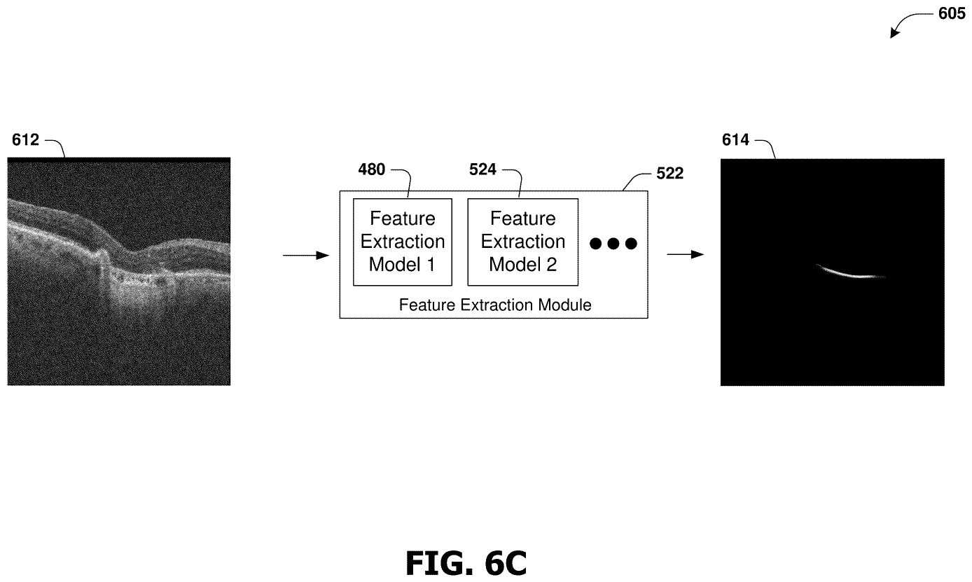

FIGS. 6A-6C illustrate example scenarios associated with determining the set of pathological features 526. FIG. 6A illustrates a first scenario 601 in which an image 602 (e.g., an OCT B-scan of at least a portion of the first eye of the first person) of the set of images 502 is processed using the feature extraction module 522 to generate a feature representation 604 corresponding to Subretinal material (SRMat) and/or subretinal hyperreflective material (SHRM). For example, the feature representation 604 may be indicative of a portion, of the first eye, where the feature extraction module 522 detected SRMat and/or SHRM in the image 602. Thus, the set of pathological features 526 may comprise SRMat and/or SHRM represented by the feature representation 604. The feature representation 604 may be generated using one or more models (e.g., the first trained feature extraction model 480), of the plurality of feature extraction models, that are trained on training information identifying segments corresponding to SRMat and/or SHRM.

FIG. 6B illustrates a second scenario 603 in which an image 608 (e.g., an OCT B-scan of at least a portion of the first eye of the first person) of the set of images 502 is processed using the feature extraction module 522 to generate a feature representation 610 corresponding to fluid (e.g., at least one of cystic fluid, general fluid, sub-RPE fluid, etc.). For example, the feature representation 610 may be indicative of a portion, of the first eye, where the feature extraction module 522 detected fluid (e.g., at least one of cystic fluid, general fluid, sub-RPE fluid, etc.) in the image 602. Thus, the set of pathological features 526 may comprise fluid (e.g., at least one of cystic fluid, general fluid, sub-RPE fluid, etc.) represented by the feature representation 610. The feature representation 610 may be generated using one or more models (e.g., the first trained feature extraction model 480), of the plurality of feature extraction models, that are trained on training information identifying segments corresponding to fluid (e.g., at least one of cystic fluid, general fluid, sub-RPE fluid, etc.).

FIG. 6C illustrates a third scenario 605 in which an image 612 (e.g., an OCT B-scan of at least a portion of the first eye of the first person) of the set of images 502 is processed using the feature extraction module 522 to generate a feature representation 614 corresponding to GA hypertransmission. For example, the feature representation 614 may be indicative of a portion, of the first eye, where the feature extraction module 522 detected GA hypertransmission in the image 612. Thus, the set of pathological features 526 may comprise GA hypertransmission represented by the feature representation 614. The feature representation 614 may be generated using one or more models (e.g., the first trained feature extraction model 480), of the plurality of feature extraction models, that are trained on training information identifying segments corresponding to GA hypertransmission.

Presence of a pathological feature in a region of the first eye of the first person may impact at-risk area segmentation associated with the region. In some examples, the trained at-risk area identification model 420 is configured to generate the at-risk area segmentation 504 based upon the set of pathological features 526 output by the feature extraction module 522.

Accordingly, the at-risk area segmentation 504 may provide segments that are more accurate representations of at-risk areas of the first eye by way of taking detected pathological features into account when generating the at-risk area segmentation 504.

The trained at-risk area identification model 420 may generate a first version of the segment 505a corresponding to the first at-risk area of the first eye based upon one or more images of the set of images 502. The trained at-risk area identification model 420 may compare one or more positions of one or more respective features of the set of pathological features 526 with a position of the segment 505a, and/or may generate a second version (e.g., an updated version) of the segment 505a based upon the comparison (e.g., the trained at-risk area identification model 420 may modify the first version of the segment 505a based upon the comparison to generate the second version of the segment 505a). The trained at-risk area identification model 420 may generate the at-risk area segmentation 504 to comprise the second version of the segment 505a (rather than the first version of the segment 505a, for example).

In an example, the set of pathological features 526 may comprise a GA area (e.g., a lesion of complete GA) and/or the segment 505a may correspond to an at-risk area associated with attenuation of EZ and/or RPE of the first eye. A first portion of the first version of the segment 505a may overlap with the GA area of the set of pathological features 526 (which may be incorrect since the GA area has deteriorated further than an “at-risk” stage and/or has progressed to complete GA, for example). In some examples, the trained at-risk area identification model 420 may modify the segment 505a by removing the first portion of the first version of the segment 505a to generate the second version of the segment 505a that does not overlap with the GA area. Alternatively and/or additionally, the trained at-risk area identification model 420 may generate the at-risk area segmentation 504 (and/or may modify and/or update the segment 505a) based upon the second segmentation 520 output by the feature extraction module 522.

In some examples, the trained at-risk area identification model 420 may be updated (e.g., further trained) using feedback information derived from the set of pathological features 526. For example, the feedback information may be indicative of (i) a modification made to update a segment (e.g., modify the segment 505a from the first version of the segment 505a to the second version of the segment 505a) based upon a feature of the set of pathological features 526, (ii) a difference between the first version of the segment 505a and the second version of the segment 505a, and/or (iii) a correct version of the segment 505a (e.g., the second version of the segment 505a). It may be appreciated that updating and/or training the trained at-risk area identification model 420 based upon the feedback information may create a closed-loop process allowing results of feature extraction and/or interactions between feature extraction and at-risk area identification as feedback to tailor settings of the trained at-risk area identification model 420. Closed-loop control may reduce errors and produce more efficient operation of a computer system which implements the trained at-risk area identification model 420. The reduction of errors and/or the efficient operation of the computer system may improve operational stability and/or predictability of operation. Accordingly, using processing circuitry to implement closed loop control described herein may improve operation of underlying hardware of the computer system.

In some examples, the system 501 comprises an imaging evaluation module 532 (e.g., OCT evaluation module) configured to analyze one or more images (e.g., at least one of an OCT B-scan, an en face OCT image, the first image 508, etc.) of the set of images 502 to determine a set of (one or more) imaging features 534 (e.g., a set of OCT features) associated with the one or more images. In some examples, the set of imaging features 534 may comprise (i) an area of hyper-reflectivity (e.g., OCT hyper-reflectivity) in an image of the one or more images, (ii) an area of hypo-reflectivity (e.g., OCT hypo-reflectivity) in an image of the one or more images, (iii) an area (in an image of the one or more images) with a change in reflectivity (in comparison with a baseline image or another portion of the image, for example), and/or (iv) one or more other imaging features. In some examples, the trained at-risk area identification model 420 may generate the at-risk area segmentation 504 (and/or may modify and/or update the segment 505a) based upon the set of imaging features 534 output by the imaging evaluation module 532.

In some examples, the system 501 comprises an ocular evaluation module 528. The ocular evaluation module 528 may perform risk stratification (on the second set of at-risk areas indicated by the at-risk area segmentation 504, for example) based upon the at-risk area segmentation 504, the second segmentation 520, the set of imaging features 534 and/or the set of pathological features 526 to determine a set of (one or more) risk classifications associated with one, some or all of the second set of at-risk areas. For example, the set of risk classifications may comprise a first risk classification associated with the first at-risk area (represented by the segment 505a of the at-risk area segmentation 504, for example) of the second set of at-risk areas, a second risk classification associated with the second at-risk area (represented by the segment 505b of the at-risk area segmentation 504, for example) of the second set of at-risk areas, and/or one or more other risk classifications associated with one or more other at-risk areas of the second set of at-risk areas.

In some examples, the first risk classification may be indicative of (i) a first risk severity level associated with the first at-risk area, (ii) one or more first types of eye conditions associated with the first at-risk area, (iii) one or more relationships between the first at-risk area and one or more pathological features of the set of pathological features 526 (e.g., the first risk classification may be indicative of one or more overlapping pathological features that overlap with the first at-risk area, one or more pathological features that are adjacent to and/or within a threshold distance of the first at-risk area, etc.), (iv) one or more relationships between the first at-risk area and one or more ocular zones of the second set of ocular zones indicated by the second segmentation 520 (e.g., the first risk classification may be indicative of one or more overlapping ocular zones, of the second set of ocular zones, that overlap with the first at-risk area, one or more ocular zones that are adjacent to and/or within a threshold distance of the first at-risk area, etc.) and/or (v) other information associated with the first at-risk area of the first eye. Other risk classifications of the set of risk classifications may be indicative of one, some or all of the types of information provided herein with respect to the first risk classification. In some examples, the one or more first types of eye conditions may correspond to one or more eye conditions (e.g., at least one of thinning, photoreceptor loss, early stages of EZ deterioration and/or atrophy, etc.) that are present at the first at-risk area and/or one or more eye conditions that the first at-risk area is at risk of progressing to. In an example, the one or more first types of eye conditions may comprise at least one of geographic atrophy (GA), retinal toxicity, hydroxychloroquine toxicity, chloroquine toxicity, hydroxychloroquine retinopathy, chloroquine retinopathy, macular degeneration, age-related macular degeneration (AMD), wet AMD, dry AMD, diabetic retinopathy, diabetic macular edema (DME), an inherited retinal disease, an atrophic eye disease, an inflammatory infectious disease, retinitis pigmentosa, Stargardt disease and/or one or more other types of eye conditions.

In some examples, presence of a pathological feature (of the set of pathological features 526, for example) relative to an at-risk area may impact risk stratification associated with the at-risk area. The ocular evaluation module 528 may thus consider presence of the set of pathological features 526 to determine risk classifications of the set of risk classifications. For example, presence of one type of pathological feature in the first eye (and/or relative to the first at-risk area) may be reflective of the first person being at risk for one or more types of eye conditions, and/or presence of another type of pathological feature in the first eye (and/or relative to the first at-risk area) may be reflective of the first person being at risk for one or more other types of eye conditions. Alternatively and/or additionally, presence of a pathological feature (e.g., GA, drusen, etc.) that overlaps with and/or is adjacent to and/or in contact with the first at-risk area may indicate that there is a higher likelihood of progression of one or more eye conditions (e.g., the one or more first types of eye conditions) at the at-risk area compared with other regions of the first eye (and/or compared with other at-risk areas that are further away from and/or do not overlap with the pathological feature), and thus the ocular evaluation module 528 may increase a value of the first risk severity level based upon detecting presence of the pathological feature (indicated by the set of pathological features 526, for example). Accordingly, the first risk classification may be a more accurate representation of a risk severity, eye condition type, etc. associated with the first at-risk area by way of taking detected pathological features into account when generating the at-risk area segmentation 504.

In an example, the ocular evaluation module 528 may (i) compare a position of a first pathological feature of the set of pathological features 526 with a position of the first at-risk area (represented by the segment 505a of the at-risk area segmentation 504, for example) of the second set of at-risk areas, and/or (ii) determine the first risk classification of the first at-risk area based upon the comparison (and/or based upon one or more comparisons of the first at-risk area with one or more other pathological features of the set of pathological features 526). For example, the ocular evaluation module 528 may determine the first risk classification based upon whether the first pathological feature overlaps with the first at-risk area (e.g., the ocular evaluation module 528 may increase a value of the first risk severity level of the first at-risk area based upon the first at-risk area overlapping with (e.g., at least partially overlapping with) at least one of drusen, a hypertransmission defect, a hypotransmission defect, inflammatory lesion, SRMat, SHRM, EZ loss, fluid, IRF, SRF, cystic fluid, general fluid, sub-RPE fluid, a lesion, inflammatory debris, region with presence of new material and/or new lesion, region with absence of material and/or lesion, region of thinning, region with change in layer thickness, etc.). Alternatively and/or additionally, the ocular evaluation module 528 may determine the first risk classification based upon whether the first pathological feature is adjacent to and/or in contact with the first at-risk area (e.g., the ocular evaluation module 528 may increase a value of the first risk severity level of the first at-risk area based upon the first at-risk area being adjacent to and/or in contact with at least one of drusen, a hypertransmission defect, a hypotransmission defect, inflammatory lesion, SRMat, SHRM, EZ loss, fluid, IRF, SRF, cystic fluid, general fluid, sub-RPE fluid, a lesion, inflammatory debris, region with presence of new material and/or new lesion, region with absence of material and/or lesion, region of thinning, region with change in layer thickness, etc.). Alternatively and/or additionally, the ocular evaluation module 528 may (i) determine a distance between the first pathological feature and the first at-risk area, and/or (ii) determine the first risk classification based upon the distance. For example, the ocular evaluation module 528 may determine the first risk classification based upon whether the distance is less than a threshold distance (e.g., the ocular evaluation module 528 may increase a value of the first risk severity level of the first at-risk area based upon the distance between the first pathological feature and the first at-risk area being less than the threshold distance). Alternatively and/or additionally, the ocular evaluation module 528 may (i) analyze the set of pathological features 526 (e.g., compare positions of respective features of the set of pathological features 526 with a position of the first at-risk area) to identify a set of (one or more) relevant features, among the set of pathological features 526, that are determined to be relevant to the first at-risk area and/or impactful upon risk stratification of the first at-risk area (e.g., the first pathological feature may be included in the set of relevant features based upon the first pathological feature overlapping with (e.g., at least partially overlapping with), being adjacent to, being in contact with, and/or being within the threshold distance of the first at-risk area) and/or (ii) determine the first risk classification based upon the set of relevant features (such as using one or more of the techniques provided herein with respect to determining the first risk classification of the first at-risk area based upon the first pathological feature, for example).

In some examples, the ocular evaluation module 528 may determine the first risk classification based upon the second segmentation 520. In some examples, one or more metrics and/or interrelationships associated with one or more ocular zones of the second set of ocular zones may be determined based upon the second segmentation 520 (e.g., the one or more metrics may comprise one or more thicknesses and/or other dimensions of ocular zones, one or more distances between ocular zones of the second set of ocular zones, etc.), and/or may be used to determine the first risk classification.

In some examples, the ocular evaluation module 528 may determine the first risk classification based upon a drug plan associated with the first person, which may be indicative of one or more drugs taken by the first person. In an example, the ocular evaluation module 528 may determine the one or more first types of eye conditions to include hydroxychloroquine toxicity and/or hydroxychloroquine retinopathy based upon the drug plan being indicative of hydroxychloroquine. Alternatively and/or additionally, the ocular evaluation module 528 may determine the one or more first types of eye conditions to include chloroquine toxicity and/or chloroquine retinopathy based upon the drug plan being indicative of chloroquine.

In some examples, the ocular evaluation module 528 may comprise a risk classification machine learning model, which may be trained on labeled information comprising (i) a plurality of at-risk area segmentations (e.g., the at-risk area segmentation 504 and/or other at-risk area segmentations generated for a plurality of persons with various eye conditions), and/or (ii) risk classifications associated with the plurality of at-risk area segmentations (e.g., the risk classifications may indicate one or more sets of information for each of the plurality of at-risk area segmentations, such as at least one of risk severity level, one or more types of eye conditions, one or more relationships with one or more pathological features, one or more relationships with one or more ocular zones, one or more relationships with one or more imaging features, etc.). For example, the risk classification machine learning model may learn to determine at least one of a risk severity level, one or more types of eye conditions, etc. associated with an at-risk area indicated by the at-risk area segmentation 504 based upon a visual appearance of the at-risk area, a pattern of at-risk areas exhibited by the at-risk area segmentation 504 (e.g., patterns of EZ loss and/or photoreceptor loss exhibited by the at-risk area segmentation 504 may linked to diagnoses of various conditions, prognoses, etc.), the second segmentation 520 indicative of the second set of ocular zones, the set of pathological features 526 and/or the set of imaging features 534.

Alternatively and/or additionally, the ocular evaluation module 528 may perform pathological feature identification based upon the second segmentation 520 to identify one or more pathological features, and/or may determine one or more risk classifications of the set of risk classifications based upon the one or more pathological features (such as using one or more of the techniques provided herein with respect to determining the first risk classification of the first at-risk area based upon the first pathological feature, for example).

In some examples, the ocular evaluation module 528 may determine the first risk classification based upon the set of imaging features 534. In some examples, presence of an imaging feature relative to an at-risk area may impact risk stratification associated with the at-risk area. The ocular evaluation module 528 may thus consider presence of the set of imaging features 534 to determine risk classifications of the set of risk classifications. For example, presence of one type of imaging feature in the first eye (and/or relative to the first at-risk area) may be reflective of the first person being at risk for one or more types of eye conditions, and/or presence of another type of imaging feature in the first eye (and/or relative to the first at-risk area) may be reflective of the first person being at risk for one or more other types of eye conditions. Alternatively and/or additionally, presence of an imaging feature (e.g., hypo-reflectivity area, hyper-reflectivity area, etc.) that overlaps with and/or is adjacent to and/or in contact with the first at-risk area may indicate that there is a higher likelihood of progression of one or more eye conditions (e.g., the one or more first types of eye conditions) at the at-risk area compared with other regions of the first eye (and/or compared with other at-risk areas that are further away from and/or do not overlap with the imaging feature), and thus the ocular evaluation module 528 may increase a value of the first risk severity level based upon detecting presence of the imaging feature (indicated by the set of imaging features 534, for example).

Accordingly, the first risk classification may be a more accurate representation of a risk severity, eye condition type, etc. associated with the first at-risk area by way of taking detected imaging features into account when generating the at-risk area segmentation 504.

In an example, the ocular evaluation module 528 may (i) compare a position of a first imaging feature of the set of imaging features 534 with a position of the first at-risk area (represented by the segment 505a of the at-risk area segmentation 504, for example) of the second set of at-risk areas, and/or (ii) determine the first risk classification of the first at-risk area based upon the comparison (and/or based upon one or more comparisons of the first at-risk area with one or more other imaging features of the set of imaging features 534). For example, the ocular evaluation module 528 may determine the first risk classification based upon whether the first imaging feature overlaps with the first at-risk area (e.g., the ocular evaluation module 528 may increase a value of the first risk severity level of the first at-risk area based upon the first at-risk area overlapping with (e.g., at least partially overlapping with) at least one of an area of hyper-reflectivity, an area of hypo-reflectivity, an area with a change in reflectivity, etc.). Alternatively and/or additionally, the ocular evaluation module 528 may determine the first risk classification based upon whether the first imaging feature is adjacent to and/or in contact with the first at-risk area (e.g., the ocular evaluation module 528 may increase a value of the first risk severity level of the first at-risk area based upon the first at-risk area being adjacent to at least one of an area of hyper-reflectivity, an area of hypo-reflectivity, an area with a change in reflectivity, etc.).

Alternatively and/or additionally, the ocular evaluation module 528 may (i) determine a distance between the first imaging feature and the first at-risk area, and/or (ii) determine the first risk classification based upon the distance. For example, the ocular evaluation module 528 may determine the first risk classification based upon whether the distance is less than a threshold distance (e.g., the ocular evaluation module 528 may increase a value of the first risk severity level of the first at-risk area based upon the distance between the first imaging feature and the first at-risk area being less than the threshold distance). Alternatively and/or additionally, the ocular evaluation module 528 may (i) analyze the set of imaging features 534 (e.g., compare positions of respective features of the set of imaging features 534 with a position of the first at-risk area) to identify a set of (one or more) relevant imaging features, among the set of imaging features 534, that are determined to be relevant to the first at-risk area and/or impactful upon risk stratification of the first at-risk area (e.g., the first imaging feature may be included in the set of relevant imaging features based upon the first imaging feature overlapping with, being adjacent to, being in contact with, and/or being within the threshold distance of the first at-risk area) and/or (ii) determine the first risk classification based upon the set of relevant imaging features (such as using one or more of the techniques provided herein with respect to determining the first risk classification of the first at-risk area based upon the first imaging feature, for example).

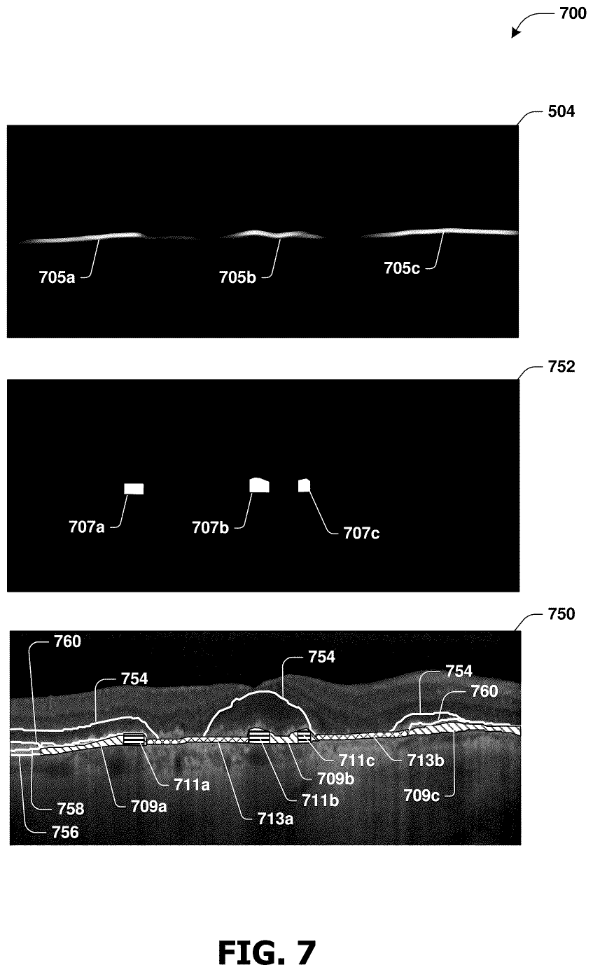

FIG. 7 illustrates example representations associated with an example scenario 700 in which the at-risk area segmentation 504 produced by the at-risk area identification model 420 comprises at-risk areas represented by segments 705a, 705b and 705c. In an example, the ocular evaluation module 528 may identify one or more first pathological areas 707a, 707b and/or 707c corresponding to one or more first pathological features. In some examples, the one or more first pathological areas 707a, 707b and/or 707c and/or the one or more first pathological features may be determined using the feature extraction module 522 (e.g., the set of pathological features 526 provided by the feature extraction module 522 may be indicative of a feature representation 752 of the one or more first pathological areas 707a, 707b and/or 707c). In an example in which the one or more first pathological features each comprise drusen, the one or more first pathological areas 707a, 707b and/or 707c and/or the one or more first pathological features may be determined using a machine learning model (e.g., a feature extraction model of the plurality of feature extraction models) that is trained (using images of drusen, for example) to identify drusen. Alternatively and/or additionally, the one or more first pathological features may comprise one or more other types of pathological features, such as at least one of a hypertransmission defect, a hypotransmission defect, inflammatory lesion, SRMat, SHRM, EZ loss, fluid, IRF, SRF, cystic fluid, general fluid, sub-RPE fluid, a lesion, inflammatory debris, region with presence of new material and/or new lesion, region with absence of material and/or lesion, region of thinning, region with change in layer thickness, etc.

Alternatively and/or additionally, the ocular evaluation module 528 may determine the one or more first pathological areas 707a, 707b and/or 707c based upon the second set of ocular zones indicated by the second segmentation 520. An at-risk segmentation profile 750 (generated by the ocular evaluation module 528, for example) associated with the example scenario 700 may be indicative of one or more segments of the second set of segments of the second segmentation 520, such as the fifth segment (shown with reference number 760) corresponding to ellipsoid zone (EZ) (e.g., the fifth ocular zone corresponds to at least a portion of EZ), the sixth segment (shown with reference number 758) corresponding to retinal pigment epithelium (RPE) (e.g., the sixth ocular zone corresponds to at least a portion of RPE), the seventh segment (shown with reference number 756) corresponding to Bruch's membrane (BM) (e.g., the seventh ocular zone corresponds to at least a portion of BM) and/or the eighth segment (shown with reference number 754) corresponding to outer nuclear layer (ONL) (e.g., the eighth ocular zone corresponds to at least a portion of ONL)).

In an example, the ocular evaluation module 528 may (i) analyze the second segmentation 520 (e.g., analyze segments 758 and 756 corresponding to RPE and BM, respectively) to measure a plurality of RPE-BM distances (which may also be referred to as RPE-BM thicknesses) at various portions of the second set of ocular zones, (ii) compare the plurality of RPE-BM distances with a first defined threshold RPE-BM distance and/or a first defined range of RPE-BM distances, (iii) determine the one or more first pathological areas based upon the comparisons, and/or (iv) mark the one or more first pathological areas as pathological in the feature representation 752. In some examples, the ocular evaluation module 528 may identify a pathological area of the one or more first pathological areas based upon a determination that an RPE-BM distance (of the plurality of RPE-BM distances) between a portion of the RPE and a portion of the BM within the pathological area is greater than the first defined threshold RPE-BM distance and/or within the first defined range of RPE-BM distances. In some examples, an RPE-BM distance of the plurality of RPE-BM distances corresponds to a distance between BM (represented by the seventh segment 756) and RPE (represented by the sixth segment 758). Accordingly, the ocular evaluation module 528 may compute the plurality of RPE-BM distances by measuring distances between BM and RPE using the sixth segment 758 and/or the seventh segment 756. Embodiments are contemplated in which one or more other types of measurements (e.g., distances, thicknesses, and/or other types of measurements other than RPE-BM distances) associated with one or more other types of ocular zones (e.g., retinal zones and/or ocular zones other than EZ and/or RPE, such as at least one of ILM, BM, ONL, POS, ELM, OPL, INL, IPL, GCL, RNFL, etc.) are computed by the ocular evaluation module 528 based upon the second segmentation 520, and the measurements are used to identify the one or more first pathological areas 707a, 707b and/or 707c.

In some examples, the first defined threshold EZ-RPE thickness may be between about 30 micrometers to about 70 micrometers (e.g., the first defined threshold EZ-RPE thickness may be about 50 micrometers) and/or other value. In some examples, the first defined range of EZ-RPE thicknesses may range from at least about 20 micrometers to at most about 200 micrometers, may range from at least about 40 micrometers to at most about 200 micrometers, and/or may be another range of values. A distance between RPE and BM exceeding the first defined threshold EZ-RPE thickness may indicate presence of drusen (e.g., significant drusen) (e.g., the one or more first pathological areas 707a, 707b and/or 707c may correspond to areas of drusen).

In some examples, the ocular evaluation module 528 may generate the at-risk segmentation profile 750 based upon the at-risk area segmentation 504, the set of risk classifications, the second segmentation 520, the set of pathological features 526 and/or the set of imaging features 534. The at-risk segmentation profile 750 may be visually representative of risk classifications (of the set of risk classifications) associated with at-risk areas, such as through use of at least one of one or more colors, patterns, shading, etc. For example, the at-risk segmentation profile 750 may display at-risk drusen areas 711a, 711b and 711c with a first pattern (horizontal stripe pattern) indicative of a at-risk drusen classification corresponding to at-risk drusen areas (of the second set of at-risk areas) that overlap with areas of drusen. For example, at-risk drusen area 711a may correspond to a region in which at least a portion of segment 705a of the at-risk area segmentation 504 overlaps with at least a portion of pathological area 707a (e.g., drusen) of the one or more first pathological areas. At-risk drusen area 711b may correspond to a region in which at least a portion of segment 705b of the at-risk area segmentation 504 overlaps with at least a portion of pathological area 707b (e.g., drusen) of the one or more first pathological areas. In some examples, presence of an at-risk drusen area may be indicative of a higher risk severity and/or an increased likelihood of progression and/or conversion to developing one or more eye conditions (e.g., GA and/or other eye condition), and thus the at-risk segmentation profile 750 may provide a healthcare professional with useful and/or clear insights that can be used to accurately and/or effectively treat the first person (by way of early detection of at-risk drusen areas and/or visual representation of the at-risk drusen areas, for example). In some examples, the at-risk segmentation profile 750 may be transmitted to a client device (e.g., at least one of a phone, a tablet, a laptop, a computer, a wearable device, a smart device, a television, any other type of computing device, hardware and/or software) and/or displayed via a graphical user interface. Alternatively and/or additionally, the at-risk segmentation profile 750 may display at-risk areas 709a, 709b and 709c that do not overlap with a pathological feature with a second pattern (diagonal stripe pattern).

Alternatively and/or additionally, the at-risk segmentation profile 750 may display GA areas 713a and 713b (e.g., areas of complete GA) with a third pattern (diamond pattern).

In some examples, the ocular evaluation module 528 analyzes the at-risk area segmentation 504 (and/or the set of risk classifications and/or the second segmentation 520 and/or the set of pathological features 526) to determine a set of (one or more) parameters. The set of parameters may be usable for diagnosing and/or treating the first person, and/or determining a stage of progression of one or more eye conditions (e.g., GA, retinal toxicity, etc.) associated with the first person. In an example, the set of parameters may comprise (i) a measure of at-risk areas of the second set of at-risk areas (e.g., an amount of area occupied by the second set of at-risk areas), (ii) a measure of at-risk areas associated with a defined risk classification (e.g., an amount of area occupied by the at-risk drusen areas associated with the at-risk drusen classification), (iii) an ellipsoid zone (EZ) integrity, (iv) a measure of retinal toxicity (e.g., hydroxychloroquine toxicity and/or chloroquine toxicity in the first person's eye), and/or (v) one or more other measures.

In some examples, the ocular evaluation module 528 may analyze the at-risk area segmentation 504 (and/or the set of risk classifications, the second segmentation 520, the set of pathological features 526 and/or the set of parameters) to diagnose the first person with one or more first eye conditions (e.g., one or more types of eye conditions indicated by risk classifications of the set of risk classifications), (ii) determine one or more likelihoods that the first person progresses to one or more second eye conditions, and/or (iii) generate a treatment plan for the first person (e.g., a treatment plan for treating the one or more first eye conditions and/or mitigating progression associated with developing the one or more second eye conditions, such as ceasing use of chloroquine and/or hydroxychloroquine based upon the first person being diagnosed with and/or at risk for hydroxychloroquine toxicity, chloroquine toxicity, hydroxychloroquine retinopathy and/or chloroquine retinopathy). In some examples, a treatment of the first person may be controlled based upon the treatment plan. In an example, the one or more first eye conditions and/or one or more second eye conditions may comprise at least one of geographic atrophy (GA), retinal toxicity, hydroxychloroquine toxicity, chloroquine toxicity, hydroxychloroquine retinopathy, chloroquine retinopathy, macular degeneration, age-related macular degeneration (AMD), wet AMD, dry AMD, diabetic retinopathy, diabetic macular edema (DME), an inherited retinal disease, an atrophic eye disease, an inflammatory infectious disease, retinitis pigmentosa, Stargardt disease and/or one or more other types of eye conditions.

In some examples, the ocular evaluation module 528 may generate an ocular report 530 based upon the at-risk area segmentation 504, the set of risk classifications, the second segmentation 520, the set of pathological features 526, the set of parameters, the one or more first eye conditions, the one or more second eye conditions, the one or more likelihoods and/or the treatment plan. For example, the ocular report 530 may comprise one or more graphical objects (e.g., one or more charts, one or more B-scans, etc.) and/or text indicative of the at-risk area segmentation 504, the set of risk classifications, the second segmentation 520, the set of pathological features 526, the set of parameters, the one or more first eye conditions, the one or more second eye conditions, the one or more likelihoods and/or the treatment plan. In an example, the ocular report 530 may comprise a representation at-risk areas (of the second set of at-risk areas) overlaid onto an image, such as a B-scan, of the first eye of the first person. In some examples, the ocular report 530 may comprise the at-risk area segmentation 504, the feature representation 752 and/or the at-risk segmentation profile 750.

In some examples, the ocular report 530 may be transmitted to a client device (e.g., at least one of a phone, a tablet, a laptop, a computer, a wearable device, a smart device, a television, any other type of computing device, hardware and/or software) and/or displayed via a graphical user interface. For example, the ocular report 530 may be displayed to one or more healthcare professionals (e.g., physician, surgeon, nurse, etc.) that are associated with the treatment of the first person. For example, the ocular report 530 may provide a healthcare professional with information that allows for early detection of at-risk areas (e.g., photoreceptor loss, EZ disruption, etc.) associated with risk of one or more eye conditions.

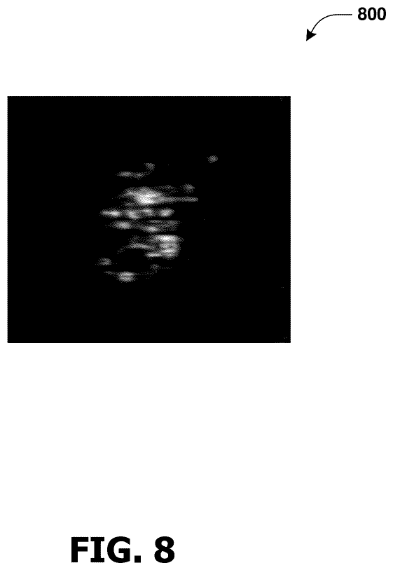

In some examples, the ocular report 530 may comprise an en face at-risk area representation 800, an example of which is shown in FIG. 8. In some examples, the trained at-risk area identification model 420 may generate a plurality of at-risk area segmentations associated with a plurality of OCT images (e.g., OCT B-scans) comprising the at-risk area segmentation 504 associated with the first image 508, a second at-risk area segmentation (not shown) associated with a second image (not shown) of the set of images 502, and/or one or more other at-risk area segmentations associated with one or more other images of the set of images 502. In some examples, the plurality of at-risk area segmentations may be assembled to generate the en face at-risk area representation 800. Each at-risk area segmentation of the plurality of at-risk area segmentations may identify one or more segments, of an image (e.g., a B-scan) of the plurality of OCT images, corresponding to one or more at-risk areas associated with a risk of one or more eye conditions. The en face at-risk area representation 800 may enable a healthcare professional to evaluate at-risk areas of the first eye over two lateral dimensions (rather than a single horizontal dimension corresponding to an OCT B-scan, for example), which may be more interpretable to the healthcare professional.

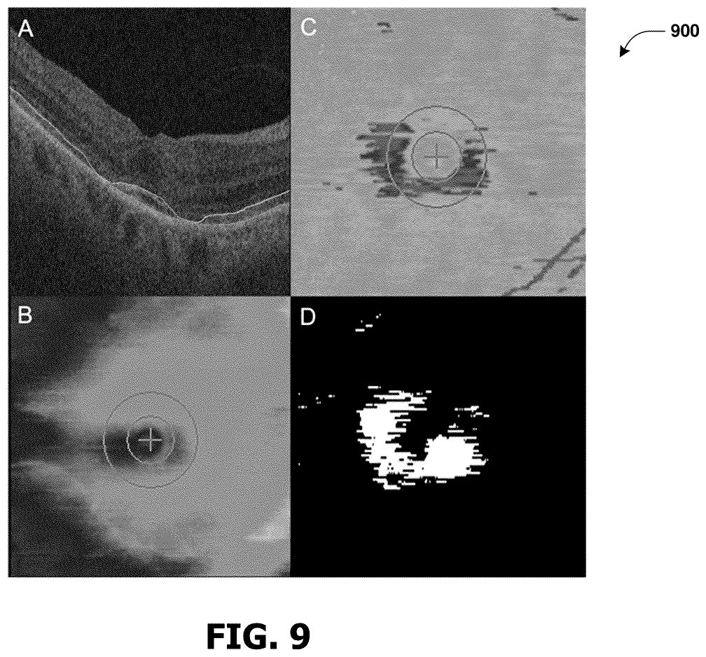

FIG. 9 illustrates an example representation 900 of at least a portion of the ocular report 530. The example representation 900 may be associated with an example of parafoveal ellipsoid zone (EZ) at-risk in toxic eye while on hydroxychloroquine (HCQ). Images A and B of the example representation 900 show OCT images of the first person while taking hydroxychloroquine as part of a drug plan. Image C of the example representation 900 shows an en face EZ-RPE mapping indicative of areas of parafoveal partial ellipsoid zone (EZ)-retinal pigment epithelium (RPE) attenuation (which may appear as a different shade and/or color than other areas of the Image C). Image D of the example representation 900 shows an en face at-risk area representation (e.g., generated using the trained at-risk area identification model 420 using one or more of the techniques provided herein with respect to generating the en face at-risk area representation 800) identifying one or more at-risk areas (e.g., at-risk areas of EZ and/or RPE of the first eye of the first person). The ocular report 530 may comprise a suggestion that the first person cease taking hydroxychloroquine as part of the drug plan.

In some examples, the ocular report 530 may be indicative of historical information associated with the first person comprising (i) one or more historical at-risk areas, risk classifications and/or pathological features, (ii) changes in at-risk areas, risk classifications and/or pathological features over time and/or (iii) comparisons between at-risk areas, risk classifications and/or pathological features associated with different times.

In some examples, each machine learning model of one, some and/or all machine learning models of the present disclosure (e.g., the trained at-risk area identification model 420, the risk classification machine learning model, a machine learning model used by the first ocular zone segmentation module 446 to perform ocular zone segmentation (for generating the first segmentation 448, for example), a machine learning model used by the second ocular zone segmentation module 506 to perform ocular zone segmentation (for generating the second segmentation 520, for example), the first trained feature extraction model 480, the second trained feature extraction model 524, one or more other machine learning models of the feature extraction module 522, etc.) may be configured for biomedical image segmentation and/or may comprise at least one of a neural network, such as a convolutional neural network (e.g., a convolutional neural network with a deep learning U-net architecture), a deep learning model, a tree-based model, a machine learning model used to perform linear regression, a machine learning model used to perform logistic regression, a decision tree model, a support vector machine (SVM), a Bayesian network model, a k-Nearest Neighbors (k-NN) model, a K-Means model, a random forest model, a machine learning model used to perform dimensional reduction, a machine learning model used to perform gradient boosting, etc.

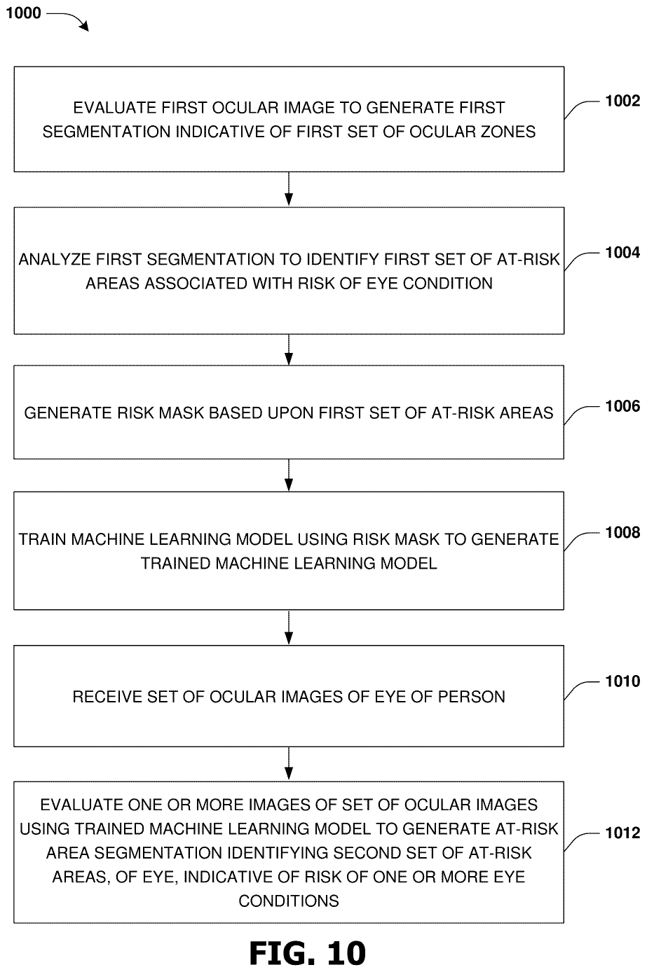

An embodiment of generating an ocular segmentation profile is illustrated by an example method 1000 of FIG. 10. At 1002, a first ocular image (e.g., the image 444) may be evaluated to generate a first segmentation (e.g., the first segmentation 448) indicative of a first set of (one or more) ocular zones. At 1004, the first segmentation may be analyzed to identify a first set of (one or more) at-risk areas associated with a risk of an eye condition. At 1006, a risk mask (e.g., the risk mask 452) may be generated based upon the first set of at-risk areas. At 1008, a machine learning model may be trained using the risk mask to generate a trained machine learning model (e.g., the trained at-risk area identification model 420). At 1010, a set of (one or more) ocular images (e.g., the set of images 502) of an eye of a person may be received. At 1012, one or more images of the set of ocular images may be evaluated using the trained machine learning model to generate an at-risk area segmentation (e.g., the at-risk area segmentation 504) identifying a second set of at-risk areas, of the eye, indicative of a risk of one or more eye conditions.

According to some embodiments, a computer-implemented method is provided. The computer-implemented method includes evaluating a first ocular image to generate a first segmentation indicative of a first set of ocular zones; analyzing the first segmentation to identify a first set of at-risk areas associated with a risk of an eye condition; generating a risk mask based upon the first set of at-risk areas; training a machine learning model using the risk mask to generate a trained machine learning model; receiving a set of ocular images of an eye of a person; and evaluating one or more images of the set of ocular images using the trained machine learning model to generate an at-risk area segmentation identifying a second set of at-risk areas, of the eye, indicative of a risk of one or more eye conditions.

According to some embodiments, the computer-implemented method includes using a feature extraction machine learning model to identify one or more pathological features of the eye based upon the set of ocular images; and performing risk stratification based upon the at-risk area segmentation and the one or more pathological features to determine a risk classification of an at-risk area of the second set of at-risk areas.

According to some embodiments, the computer-implemented method includes comparing a position of a first pathological feature of the one or more pathological features with a position of the at-risk area, wherein the risk classification of the at-risk area is determined based upon the comparison.

According to some embodiments, the computer-implemented method includes evaluating one or more images of the set of ocular images to generate a second segmentation indicative of a second set of ocular zones of the eye; analyzing the second segmentation to identify a first pathological area corresponding to a pathological feature; and performing risk stratification based upon the at-risk area segmentation and the first pathological area to determine a risk classification of an at-risk area of the second set of at-risk areas.

According to some embodiments, the computer-implemented method includes comparing a position of the first pathological area with a position of the at-risk area, wherein the risk classification of the at-risk area is determined based upon the comparison.

According to some embodiments, identifying the first pathological area indicative of the pathological feature includes: measuring distances between a first ocular zone of the second set of ocular zones and a second ocular zone of the second set of ocular zones; and comparing the distances with a defined threshold distance and/or a defined range of distances to identify the first pathological area including a portion, of the second set of ocular zones, in which a distance between the first ocular zone and the second ocular zone is: greater than the defined threshold distance; and/or within the defined range of distances.

According to some embodiments, the first ocular zone corresponds to Bruch's membrane (BM) of the eye; the second ocular zone corresponds to retinal pigment epithelium (RPE) of the eye; and the pathological feature corresponds to drusen.

According to some embodiments, identifying the first set of at-risk areas includes measuring distances between a first ocular zone of the first set of ocular zones and a second ocular zone of the first set of ocular zones; and comparing the distances with a defined threshold distance and/or a defined range of distances to identify a first at-risk area including a portion, of the first set of ocular zones, in which a distance between the first ocular zone and the second ocular zone is less than the defined threshold distance and/or within the defined range of distances.

According to some embodiments, generating the risk mask includes generating the risk mask to include a representation of the first at-risk area.

According to some embodiments, generating the risk mask includes generating the risk mask to exclude a representation of an area associated with complete geographic atrophy (GA).

According to some embodiments, the first ocular zone corresponds to Ellipsoid Zone (EZ); and the second ocular zone corresponds to retinal pigment epithelium (RPE).

According to some embodiments, an ocular zone of the first set of ocular zones corresponds to at least a portion of a retina.

According to some embodiments, an ocular zone of the first set of ocular zones corresponds to at least a portion of an outer retinal zone.

According to some embodiments, the computer-implemented method includes performing risk stratification based upon the at-risk area segmentation to determine a risk classification of an at-risk area of the second set of at-risk areas, wherein the risk classification is indicative of risk severity level and/or a type of eye condition associated with the at-risk area.

According to some embodiments, the computer-implemented method includes generating an ocular report indicative the at-risk area segmentation; and/or the risk classification; and providing the ocular report to a device for display.

According to some embodiments, the type of eye condition comprises geographic atrophy (GA); macular degeneration; age-related macular degeneration (AMD); wet AMD; dry AMD; diabetic retinopathy; diabetic macular edema (DME); an inherited retinal disease; an atrophic eye disease; an inflammatory infectious disease; retinitis pigmentosa; Stargardt disease; retinal toxicity; hydroxychloroquine toxicity; chloroquine toxicity; hydroxychloroquine retinopathy; and/or chloroquine retinopathy.

According to some embodiments, an ocular zone of the first set of ocular zones corresponds to: at least a portion of an internal limiting membrane (ILM); at least a portion of a Bruch's membrane (BM); at least a portion of a retinal pigment epithelium (RPE); at least a portion of an outer nuclear layer (ONL); at least a portion of an ellipsoid zone (EZ); at least a portion of photoreceptor outer segments (POS); at least a portion of an external limiting membrane (ELM); at least a portion of an outer plexiform layer (OPL); at least a portion of an inner nuclear layer (INL); at least a portion of an inner plexiform layer (IPL); at least a portion of a ganglion cell layer (GCL); and/or at least a portion of a retinal nerve fiber layer (RNFL).

According to some embodiments, a non-transitory computer-readable medium is provided. The non-transitory computer-readable medium stores instructions that when executed perform operations including evaluating a first ocular image to generate a first segmentation indicative of a first set of ocular zones; analyzing the first segmentation to identify a first set of at-risk areas associated with a risk of an eye condition; generating a risk mask based upon the first set of at-risk areas; training a machine learning model using the risk mask to generate a trained machine learning model; receiving a set of ocular images of an eye of a person; and evaluating one or more images of the set of ocular images using the trained machine learning model to generate an at-risk area segmentation identifying a second set of at-risk areas, of the eye, indicative of a risk of one or more eye conditions.

According to some embodiments, the operations include using a feature extraction machine learning model to identify one or more pathological features of the eye based upon the set of ocular images; and performing risk stratification based upon the at-risk area segmentation and the one or more pathological features to determine a risk classification of an at-risk area of the second set of at-risk areas.

According to some embodiments, a computing device is provided. The computing device includes a processor; and memory comprising processor-executable instructions that when executed by the processor cause performance of operations. The operations comprise evaluating a first ocular image to generate a first segmentation indicative of a first set of ocular zones; analyzing the first segmentation to identify a first set of at-risk areas associated with a risk of an eye condition; generating a risk mask based upon the first set of at-risk areas; training a machine learning model using the risk mask to generate a trained machine learning model; receiving a set of ocular images of an eye of a person; and evaluating one or more images of the set of ocular images using the trained machine learning model to generate an at-risk area segmentation identifying a second set of at-risk areas, of the eye, indicative of a risk of one or more eye conditions.

According to some embodiments, the operations include using a feature extraction machine learning model to identify one or more pathological features of the eye based upon the set of ocular images; and performing risk stratification based upon the at-risk area segmentation and the one or more pathological features to determine a risk classification of an at-risk area of the second set of at-risk areas.

According to some embodiments, a method including at least one aspect as described in the present disclosure and/or shown in the figures.

According to some embodiments, a method including plural aspects as described in the present disclosure and/or shown in the figures.

According to some embodiments, a system including at least one aspect as described in the present disclosure and/or shown in the figures.

According to some embodiments, a system including plural aspects as described in the present disclosure and/or shown in the figures.

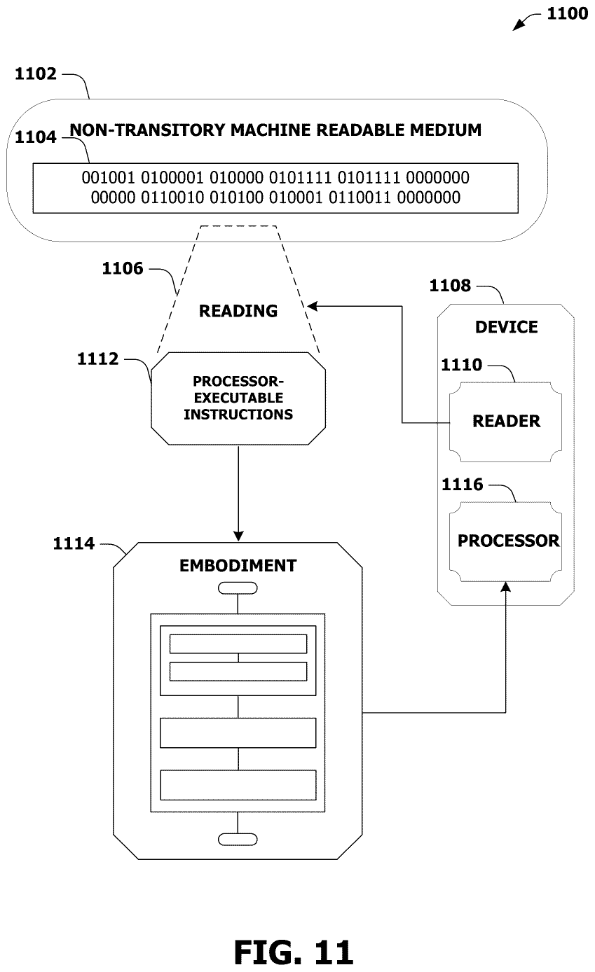

FIG. 11 is an illustration of a scenario 1100 involving an example non-transitory machine readable medium 1102. The non-transitory machine readable medium 1102 may comprise processor-executable instructions 1112 that when executed by a processor 1116 cause performance (e.g., by the processor 1116) of at least some of the provisions herein (e.g., embodiment 1114).

The non-transitory machine readable medium 1102 may comprise a memory semiconductor (e.g., a semiconductor utilizing static random access memory (SRAM), dynamic random access memory (DRAM), and/or synchronous dynamic random access memory (SDRAM) technologies), a platter of a hard disk drive, a flash memory device, or a magnetic or optical disc (such as a compact disc (CD), digital versatile disc (DVD), or floppy disk).

The example non-transitory machine readable medium 1102 stores computer-readable data 1104 that, when subjected to reading 1106 by a reader 1110 of a device 1108 (e.g., a read head of a hard disk drive, or a read operation invoked on a solid-state storage device), express the processor-executable instructions 1112.

In some embodiments, the processor-executable instructions 1112, when executed, cause performance of operations, such as at least some of the example method 1000 of FIG. 10, for example. In some embodiments, the processor-executable instructions 1112 are configured to cause implementation of a system, such as at least some of the example machine learning model training system 401 of FIGS. 4A-4E and/or the example system 501 of FIG. 5, for example.

As used in this application, “component,” “module,” “system”, “interface”, and/or the like are generally intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers.

Unless specified otherwise, “first,” “second,” and/or the like are not intended to imply a temporal aspect, a spatial aspect, an ordering, etc.

Rather, such terms are merely used as identifiers, names, etc. for features, elements, items, etc. For example, a first object and a second object generally correspond to object A and object B or two different or two identical objects or the same object.

Moreover, “example” is used herein to mean serving as an instance, illustration, etc., and not necessarily as advantageous. As used herein, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, and/or variants thereof are used in either the detailed description or the claims, such terms are intended to be inclusive in a manner similar to the term “comprising”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of the claims.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, many modifications may be made to this configuration without departing from the scope or spirit of the claimed subject matter.

Various operations of embodiments are provided herein. In an embodiment, one or more of the operations described may constitute computer readable instructions stored on one or more computer and/or machine readable media, which if executed will cause the operations to be performed. The order in which some or all of the operations are described should not be construed as to imply that these operations are necessarily order dependent. Alternative ordering will be appreciated by one skilled in the art having the benefit of this description. Further, it will be understood that not all operations are necessarily present in each embodiment provided herein.

Also, it will be understood that not all operations are necessary in some embodiments.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

1. A computer-implemented method, comprising:
evaluating a first ocular image to generate a first segmentation indicative of a first set of ocular zones;
analyzing the first segmentation to identify a first set of at-risk areas associated with a risk of an eye condition;
generating a risk mask based upon the first set of at-risk areas;
training a machine learning model using the risk mask to generate a trained machine learning model;
receiving a set of ocular images of an eye of a person; and
evaluating one or more images of the set of ocular images using the trained machine learning model to generate an at-risk area segmentation identifying a second set of at-risk areas, of the eye, indicative of a risk of one or more eye conditions.

2. The computer-implemented method of claim 1, comprising:
using a feature extraction machine learning model to identify one or more pathological features of the eye based upon the set of ocular images; and
performing risk stratification based upon the at-risk area segmentation and the one or more pathological features to determine a risk classification of an at-risk area of the second set of at-risk areas.

3. The computer-implemented method of claim 2, comprising:
comparing a position of a first pathological feature of the one or more pathological features with a position of the at-risk area, wherein the risk classification of the at-risk area is determined based upon the comparison.

4. The computer-implemented method of claim 1, comprising:
evaluating one or more images of the set of ocular images to generate a second segmentation indicative of a second set of ocular zones of the eye;
analyzing the second segmentation to identify a first pathological area corresponding to a pathological feature; and
performing risk stratification based upon the at-risk area segmentation and the first pathological area to determine a risk classification of an at-risk area of the second set of at-risk areas.

5. The computer-implemented method of claim 4, comprising:
comparing a position of the first pathological area with a position of the at-risk area, wherein the risk classification of the at-risk area is determined based upon the comparison.

6. The computer-implemented method of claim 4, wherein:
identifying the first pathological area corresponding to the pathological feature comprises:
measuring distances between a first ocular zone of the second set of ocular zones and a second ocular zone of the second set of ocular zones; and
comparing the distances with at least one of a defined threshold distance or a defined range of distances to identify the first pathological area comprising a portion, of the second set of ocular zones, in which a distance between the first ocular zone and the second ocular zone is at least one of:
greater than the defined threshold distance; or
within the defined range of distances.

7. The computer-implemented method of claim 6, wherein:
the first ocular zone corresponds to Bruch's membrane (BM) of the eye;
the second ocular zone corresponds to retinal pigment epithelium (RPE) of the eye; and
the pathological feature corresponds to drusen.

8. The computer-implemented method of claim 1, wherein:
identifying the first set of at-risk areas comprises:
measuring distances between a first ocular zone of the first set of ocular zones and a second ocular zone of the first set of ocular zones; and
comparing the distances with at least one of a defined threshold distance or a defined range of distances to identify a first at-risk area comprising a portion, of the first set of ocular zones, in which a distance between the first ocular zone and the second ocular zone is at least one of:
less than the defined threshold distance; or
within the defined range of distances.

9. The computer-implemented method of claim 8, wherein:
generating the risk mask comprises generating the risk mask to include a representation of the first at-risk area.

10. The computer-implemented method of claim 8, wherein:
generating the risk mask comprises generating the risk mask to exclude a representation of an area associated with complete geographic atrophy (GA).

11. The computer-implemented method of claim 8, wherein:
the first ocular zone corresponds to Ellipsoid Zone (EZ); and
the second ocular zone corresponds to retinal pigment epithelium (RPE).

12. The computer-implemented method of claim 1, wherein:
an ocular zone of the first set of ocular zones corresponds to at least a portion of a retina.

13. The computer-implemented method of claim 1, wherein:
an ocular zone of the first set of ocular zones corresponds to at least a portion of an outer retinal zone.

14. The computer-implemented method of claim 1, comprising:
performing risk stratification based upon the at-risk area segmentation to determine a risk classification of an at-risk area of the second set of at-risk areas, wherein the risk classification is indicative of at least one of risk severity level or a type of eye condition associated with the at-risk area.

15. The computer-implemented method of claim 14, comprising:
generating an ocular report indicative of at least one of:
the at-risk area segmentation; or
the risk classification; and
providing the ocular report to a device for display.

16. The computer-implemented method of claim 14, wherein the type of eye condition comprises at least one of:
geographic atrophy (GA);
macular degeneration;
age-related macular degeneration (AMD);
wet AMD;
dry AMD;
diabetic retinopathy;
diabetic macular edema (DME);
an inherited retinal disease;
an atrophic eye disease;
an inflammatory infectious disease;
retinitis pigmentosa;
Stargardt disease;
retinal toxicity;
hydroxychloroquine toxicity;
chloroquine toxicity;
hydroxychloroquine retinopathy; or
chloroquine retinopathy.

17. The computer-implemented method of claim 1, wherein:
an ocular zone of the first set of ocular zones corresponds to at least a portion of at least one of:
an internal limiting membrane (ILM);
a Bruch's membrane (BM);
a retinal pigment epithelium (RPE);
an outer nuclear layer (ONL);
an ellipsoid zone (EZ);
photoreceptor outer segments (POS);
an external limiting membrane (ELM);
an outer plexiform layer (OPL);
an inner nuclear layer (INL);
an inner plexiform layer (IPL);
a ganglion cell layer (GCL); or
a retinal nerve fiber layer (RNFL).

18. A non-transitory computer-readable medium storing instructions that when executed perform operations comprising:
evaluating a first ocular image to generate a first segmentation indicative of a first set of ocular zones;
analyzing the first segmentation to identify a first set of at-risk areas associated with a risk of an eye condition;
generating a risk mask based upon the first set of at-risk areas;
training a machine learning model using the risk mask to generate a trained machine learning model;
receiving a set of ocular images of an eye of a person; and
evaluating one or more images of the set of ocular images using the trained machine learning model to generate an at-risk area segmentation identifying a second set of at-risk areas, of the eye, indicative of a risk of one or more eye conditions.

19. The non-transitory computer-readable medium of claim 18, the operations comprising:
using a feature extraction machine learning model to identify one or more pathological features of the eye based upon the set of ocular images; and
performing risk stratification based upon the at-risk area segmentation and the one or more pathological features to determine a risk classification of an at-risk area of the second set of at-risk areas.

20. A computing device comprising:
a processor; and
memory comprising processor-executable instructions that when executed by the processor cause performance of operations, the operations comprising:
evaluating a first ocular image to generate a first segmentation indicative of a first set of ocular zones;
analyzing the first segmentation to identify a first set of at-risk areas associated with a risk of an eye condition;
generating a risk mask based upon the first set of at-risk areas;
training a machine learning model using the risk mask to generate a trained machine learning model;
receiving a set of ocular images of an eye of a person; and
evaluating one or more images of the set of ocular images using the trained machine learning model to generate an at-risk area segmentation identifying a second set of at-risk areas, of the eye, indicative of a risk of one or more eye conditions.

21-26. (canceled)
